(19) United States
(12) Patent Application Publication
(10) Pub. No.: US 2011/0229485 A1
(43) Pub. Date: Sep. 22, 2011

(54) INHIBITION OF THE NT-3:TRKC BOUND AND ITS APPLICATION TO THE TREATMENT OF CANCER SUCH AS NEUROBLASTOMA

(75) Inventors: Patrick Etienne Roger Mehlen, Serezin Du Rhone, (FR); Servane Marie-séverine Tauszig-Delamasure, Lyon (FR); Céline Jacqueline-Andrée Delloye, Lyon (FR); Jimena Bouzas-Rodriguez, Lyon (FR)

(73) Assignees: CENTRE NATIONAL DE LA RECHERCHE SCIENTFIQUE (CNRS), Paris (FR); CENTRE LEON BERARD, Lyon (FR); ECOLE NORMALE SUPERIEURE DE LYON, Lyon (FR); UNIVERSITE CLAUDE BERNARD LYON 1, Villeurbanne (FR)

(21) Appl. No.: 12/993,663

(22) PCT Filed: May 22, 2009
(86) PCT No.: PCT/EP09/56253

§ 371 Date: May 19, 2011

Related U.S. Application Data
(60) Provisional application No. 61/055,052, filed on May 21, 2008.

(51) Int. Cl. A61K 39/395; G01N 33/53; G01N 33/567; C12Q 1/68; C07K 14/435; C07K 16/30; C07H 21/02; A61K 38/17; A61P 35/00; A61P 35/04; A61K 31/713

(52) U.S. Cl. 424/143.1; 435/7.92; 436/501; 435/7.2; 435/6.12; 530/350; 530/388.22; 530/389.7; 536/24.5; 514/19.3; 514/19.8; 424/174.1; 514/44 A

(57) ABSTRACT

The subject matter of the present disclosure relates to an in vitro method for the screening of anti-cancer compounds based on the capacity for these compounds to interact with neurotrophin 3 (NT-3 or NT3), to the extracellular domain, or TrkC receptor and/or to inhibit the dimerization of the intracellular domain of the TrkC receptor expressed in tumor cells, particularly in neuroblastoma. The disclosure also relates to a method for predicting the presence of metastatic cancer or a bad prognosis cancer, or for determining the efficiency of an anti-cancer treatment based on the measuring of the expression level of neurotrophin 3. The disclosure further comprises kits and compounds as a medicament for the treatment of neuroblastoma or cancer overexpressing neurotrophin 3 by the tumor cells.

### CROSS-REFERENCE TO RELATED APPLICATIONS

This application is a National Phase Entry of International Application No. PCT/EP2009/056253, filed on May 22, 2009, which claims priority to U.S. Application Ser. No. 61/055,052, filed on May 21, 2008, both of which are incorporated by reference herein.

### BACKGROUND AND SUMMARY

The subject matter of the present invention relates to an in vitro method for the screening of anti-cancer compounds based on the capacity for these compounds to interact with neurotrophin 3 (NT-3 or NT3), to the extracellular domain or TrkC receptor and/or to inhibit the dimerization of the intracellular domain of the TrkC receptor expressed in tumor cells, particularly in neuroblastoma. The invention also relates to a method for predicting the presence of metastatic cancer or a poor prognosis cancer, or for determining the efficiency of an anti-cancer treatment based on the measuring of the expression level of neurotrophin 3. The invention further comprises kits and compounds as a medicament for the treatment of neuroblastoma or cancer overexpressing neurotrophin 3 by the tumor cells.

The TrkC/NT-3 receptor/ligand pair is believed to be part of the classic neurotrophic theory claiming that neuronal death occurs by default when neurotrophic factors become limited, through loss of survival signals. Here, we show that TrkC is a dependence receptor and, as such, induces caspase-dependent apoptotic death in the absence of NT-3 in immortalized cells, a proapoptotic activity inhibited by the presence of NT-3. This proapoptotic activity of TrkC relies on the caspase-mediated cleavage of the intracellular domain of TrkC, which permits the release of a proapoptotic fragment. This fragment induces apoptosis through a caspase-9-dependent mechanism. Finally, we show that the death of dorsal root ganglion (DRG) neurons provoked by NT-3 withdrawal is inhibited when TrkC-proapoptotic activity is antagonized. Thus, the death of neurons upon disappearance of NT-3 is not only due to a loss of survival signals but also to the active proapoptotic activity of the unbound TrkC dependence receptor.

The classic neurotrophic theory usually proposes that neuronal survival depends on neurotrophic factors, such as neurotrophins (1, 2). This theory also claims that death triggered when these neurotrophic factors become limited is due to a loss of survival signals (3). Neurotrophins include NGF, BDNF, NT-3, and NT-4/5 (2). These proteins have been shown to be crucial for the development of the nervous system, especially by controlling the massive developmental loss of neurons that are produced in excess and that fail to adequately connect their targets. The current neurotrophic model holds that the main neurotrophin receptors, TrkA, TrkB, and TrkC, generate survival signals via the PI3K/Akt and Ras/MEK/MAPK pathways upon neurotrophin binding (3). This binding is thought to inhibit the naturally occurring apoptotic death of neurons. However, a weakness in this theory is that the molecular nature of the “default apoptotic state” of the developing neurons is not understood. One mechanism could be that a death signal is actively generated in the neurons. When bound by the ligand, death receptors of the tumor necrosis receptor family trigger caspase activation and death of many cell types, but there is little evidence of their involvement in the nervous system. However, recent observations support that, depending on the availability of the ligand, some receptors initiate two completely opposite signaling pathways: in the presence of ligand, these receptors transduce a positive signal of differentiation, guidance, or survival, whereas in the absence of ligand, they induce an active process of apoptotic cell death. These receptors, called dependence receptors, include p75 ntr, DCC (deleted in colorectal cancer), UNC5H, Patched, Neogenin, and the tyrosine kinase receptor RET (4-10). The proapoptotic activity of these receptors, observed in the absence of their respective ligand, has been speculated to be important to dictate the adequate territories of neuron migration or localization during the development of the nervous system but also more importantly to regulate tumor growth in adult. This activity has been exemplified in vivo with the dependence receptor Patched and the survival of neuroepithelial cells in the developing spinal cord (4) as well as for the netrin-1 receptors DCC and/or UNC5H in colorectal tumorigenesis (5). Along this latter case, netrin-1 receptor have been shown to be tumor suppressor inhibiting tumor progression by inducing apoptosis of tumor cell growing in setting of ligand limitation—i.e., primary tumor growth or metastasis—.

The inventors provide evidence that the protein tyrosine kinase receptor TrkC, a main cognate receptor for NT-3, is also a dependence receptor and that this dependence receptor TrkC behaves as a tumor suppressor in neuroblastoma by regulating apoptosis. A selective advantage for aggressive tumor cell is then to either downregulate TrkC (along this line TrkC expression is associated with good prognosis neuroblatoma) or as hypothesized by the inventors an autocrine over-expression of NT-3. This question is important not only for basic knowledge, but is crucial for therapy: indeed, inhibiting the extracellular interaction between neurothophin 3 and TrkC dependence receptor in these NT-3 high tumors represents an appealing strategy to trigger tumor regression for tumors related to an overexpression of NT-3 or exhibiting a high ratio NT-3/TrkC. It is particular desirable to provide simple and consistent means for identifying and characterizing new compounds which can be used for the treatment of such cancer.

Surprisingly, the inventors have demonstrated that TrkC induces apoptosis in NT-3 expressing tumor cells such as neuroblastoma cells, when incubated in presence of a compound capable of antagonizing TrkC-NT3 bound. Preliminary results showed reduced primary tumor development, and suppression of metastasis and demonstrate that such compounds can be used in therapy to trigger death of metastasic tumor, and thus as potential drug for the treatment and/or the prevention of cancer which results from an overexpression of NT-3 or which exhibits a high ratio NT3:TrkC.

In a first aspect, the present invention is directed to an in vitro method for selecting a compound for the prevention or the treatment of cancer, wherein said method comprises the following steps of:

a) having a medium containing neurotrophin 3, or a fragment thereof, and a TrkC receptor, or a fragment thereof, wherein:

said neurotrophin 3, or a fragment thereof, and said TrkC receptor, or a fragment thereof, is able to specifically interact together to form a binding pair, and/or

said neurotrophin 3, or a fragment thereof, is able to induce the dimerization or multimerization of said TrkC receptor, or a fragment thereof, particularly the intracellular domain of said TrkC receptor;

b) contacting said medium with the compound to be tested;
c) measuring the inhibition of the interaction between neurotrophin 3, or a fragment thereof, and said TrkC receptor, or a fragment thereof, and/or

determine whether said compound inhibit the dimerization or multimerization of said TrkC receptor, or a fragment thereof, particularly the dimerization of the intracellular domain of said TrkC receptor; and

d) selecting said compound if:

the measuring in step c) demonstrates a significantly inhibition of the interaction between neurotrophin 3, or a fragment thereof, and TrkC receptor, or a fragment thereof, in presence of said compound, and/or

the determination in step c) demonstrates a significantly inhibition of the dimerization or multimerization of said TrkC receptor, or a fragment thereof, in presence of said compound, particularly the dimerization of the intracellular domain of said TrkC receptor.

By the terms interaction between neurotrophin 3 and its TrkC receptor, it is intended to designate in the present application the interaction which result to the selective advantage for tumor cells to escape neurotrophin 3 dependence receptors induced apoptosis, preferably due to elevated neurotrophin 3 level. So, the inhibition of this interaction can be obtained for example by the complete or partial inhibition of the binding of neurotrophin 3 to its TrkC receptor, notably in presence of a competitive ligand (such as an antibody, a monoclonal or a polyclonal antibody which is directed to the extracellular membrane domain of said TrkC receptor), or in presence of a compound able to form a specific complex with the neurotrophin 3 (such as a soluble extracellular membrane domain of its TrkC receptor, or part thereof).

In a preferred embodiment, the method according to the present invention is characterized in that said cancer to be prevented or treated is a cancer wherein tumoral cells express or overexpress neurotrophin 3 or exhibit a high ratio NT3:TrkC. In another preferred embodiment, the method according to the present invention is characterized in that said cancer to be prevented or treated is neuroblastoma or breast cancer. In another preferred embodiment, the method according to the present invention is characterized in that said cancer to be prevented or treated is a metastatic, an aggressive cancer or a bad prognosis cancer.

In another preferred embodiment, the method according to the present invention is characterized in that at step a):

said TrkC receptor fragment comprises or is the extracellular domain of the TrkC receptor, or part thereof able to interact with neurotrophin 3, preferably the extracellular fragment which comprises at least the N-terminal fragment containing the first 429 amino acid residues of the humant TrkC or of a natural variant thereof having at least 95% identity with the amino acid sequence depicted in Genbank A. N. AAB33111 dated Jul. 27, 1995; and/or

said TrkC receptor fragment comprises or is the intracellular domain of the TrkC receptor, or part thereof able to dimerize or multimerize in presence of neurotrophin 3.

In another preferred embodiment, the method according to the present invention is characterized in that said neurotrophin 3 or/and said TrkC receptor are from mammal, particularly from mouse, rat or human, preferably human. In another preferred embodiment, the method according to the present invention is characterized in that said neurotrophin 3 or/and said TrkC receptor and/or the compound to be tested is labelled by a marker able to be directly or indirectly measured.

In another preferred embodiment, the method according to the present invention is characterized in that at step c):

the measure of the inhibition of the interaction between neurotrophin 3, or a fragment thereof, and said TrkC receptor, or a fragment thereof, is carried out by immunoassay (particularly by ELISA or by Immunoradiometric Assay (IRMA)), by Scintillation Proximity Assay (SPA) or by Fluorescence Resonance Energy Transfer (FRET); and/or

the dimerization or multimerization, or its inhibition, of said TrkC receptor, or fragment thereof, particularly the intracellular domain, is carried out by immunoprecipitation or FRET.

In another particular preferred embodiment, the method according to the present invention is characterized in that at step a) said medium contains cells which express at their surface membrane an endogenous or a recombinant TrkC receptor, particularly a recombinant extracellular domain of said TrkC receptor.

In a preferred embodiment, said recombinant TrkC receptor also comprises the intracellular domain of said TrkC receptor. In another particular preferred embodiment, the method according to the present invention is characterized in that at step a) said medium contains tumoral cells, which express endogenously said TrkC receptor at their membrane surface and which express or overexpress neurotrophin 3, and wherein at step c) the inhibition of the interaction between neurotrophin 3 and its TrkC receptor in presence of the compound to be tested, is measured by the apoptosis or cells death induced by the presence of the compound to be tested, preferably analysed using the trypan blue staining method as indicated in the examples below. In a preferred embodiment said tumoral cells are selected from the group consisting of neuroblastoma established cell lines, such as CLB-Ge1 or IMR32 cells line.

The present invention is also directed to an in vitro method for selecting a compound for the prevention or the treatment of cancer, wherein said method comprises the following steps of:

a) having a medium containing a mammal cell expressing an endogenous or a recombinant TrkC receptor, or a fragment thereof comprising at least its intracellular domain, preferably a tumor cell, more preferably a cell presenting dimerization or multimerization of its TrkC receptor intracellular domain or a cell wherein its TrkC receptor intracellular domain is able to dimerize or multimere in presence of neurotrophin 3;
b) contacting said medium with the compound to be tested, optionally the medium further containing neurotrophin 3, or a fragment thereof able to interact with the extracellular domain of the TrkC receptor;
c) determine whether the dimerization or multimerization of said TrkC receptor intracellular domain is inhibited in presence of said compound to be tested;
d) optionally, determine (for example by the blue trypan method) whether the presence of the compound to be tested induces the cell death of said mammal cell; and
e) selecting said compound if the determination in step c) demonstrates a significantly inhibition of the dimerization or multimerization of the intracellular domain of said TrkC receptor and/or if the determination in step d) demonstrates the cell death of said mammal cell.

In a second aspect, the present invention is directed to an in vitro method for predicting the presence of a metastatic cancer or an aggressive cancer, particularly neuroblastoma having a bad prognosis, in a patient having a primary tumor from a biopsy of said patient containing primary tumors cells, said method comprising the following step of:

a) measuring of the neurotrophin 3 expression level in said biopsy or the ratio NT3:TrkC. In a preferred embodiment, the method for predicting according to the present invention is characterized in that at step a) wherein an increase of the neurotrophin 3 expression level in said biopsy, compared with expression of neurotrophin 3 in non-metastatic primary tumor biopsies or in non-aggressive cancer biopsies is significant of the presence of a metastatic cancer or an aggressive cancer. In a more preferred embodiment, the method for predicting according to the present invention is characterized in that a ratio superior to 2, preferably to 2.5, to 3, to 3.5, to 4, to 4.5 and to 5, between neurotrophin 3 expression in the biopsy to be tested and in the non-metastatic or non-aggressive reference biopsy is significant of the presence of a metastatic or an aggressive cancer.

In a third aspect, the present invention is directed to a method for determining in vitro the efficiency of an anti-cancer treatment for a patient or for in vitro selecting patients who are susceptible to respond to a specific anti-cancer treatment based on the inhibition of the NT-3:TrkC bound, said method comprising the following step of:

(a) obtaining a primary tumor biopsy of said treated patient; and

(b) measuring of the neurotrophin 3 expression level in said biopsy,

wherein the efficiency of said anti-cancer treatment is correlated with the decrease of the amount of the neurotrophin 3 expression level measured in said biopsy, or
wherein the selected patients who are susceptible to respond to said specific anti-cancer treatment are patients wherein the amount of the neurotrophin 3 expression level measured in their biopsy before the treatment is significantly superior to the amount of the neurotrophin 3 expression level of a control patient, and, optionally, wherein the neurotrophin 3 expression level has been decreased after said specific treatment.

In a preferred embodiment, the method for determining in vitro the efficiency of an anti-cancer treatment for a patient or for selecting patients who responds to a specific anti-cancer treatment, is characterized in that said cancer induced an overexpression of neurotrophin 3 and/or is a metastatic or an aggressive cancer. In a preferred embodiment, the method for prediction or for determining in vitro the efficiency of an anti-cancer treatment for a patient is characterized in that the measured neurotrophin 3 expression product is the RNA encoding neurotrophin 3, particularly measured by a quantitative real time reverse PCR method, or in that the expression level of neurotrophin 3 which is measured is the measure of the neurotrophin 3 protein level, particularly by a method using specific antibodies able to specifically recognize said neurotrophin 3 protein. In a preferred embodiment, the method for prediction or for determining in vitro the efficiency of an anti-cancer treatment for a patient is characterized in that the primary tumor is a primary tumor of a cancer selected from the group consisting cancer overexpressing NT-3, or exhibiting a high ratio NT3:TrkC, preferably neuroblastoma or breast cancer.

In another aspect, the present invention is directed to a kit for the selection of a compound for the prevention or the treatment of cancer, wherein said kit comprises:

a TrkC receptor protein, or a fragment thereof able to specifically interact with the neurotrophin 3 protein to form a binding pair, preferably recombinant protein; and

neurotrophin 3 protein, or a fragment thereof able to specifically interact with said TrkC receptor protein to form a binding pair, preferably recombinant protein.

Said TrkC receptor being also preferably selected from the group of TrkC, preferably from mammal such as from mouse, rat or human.

In a preferred embodiment, said kit comprises:

tumoral cells which express TrkC receptor and which express or overexpress neurotrophin 3, particularly cells from metastatic tumoral cell line, preferably selected from the group consisting neuroblastoma cell line, such as CLB-Ge1 or IMR32 cell line.

In another aspect, the present invention comprises a compound selected from the group consisting of:

a compound comprising an extracellular domain of TrkC receptor or fragment thereof able to specifically inhibit the interaction between the neurotrophin 3 and said TrkC receptor, and/or able to inhibit the dimerization or multimerization of said TrkC receptor, or a fragment thereof, particularly to inhibit the intracellular domain of said TrkC receptor;

a monoclonal (which can be humanized) or polyclonal antibody directed specifically against neurotrophin 3 or TrkC receptor, particularly directed to the extracellular domain of said TrkC receptor or to the neurotrophin 3 fragment able to interact with the extracellular domain of said TrkC receptor;

a soluble extracellular domain of said TrkC receptor capable of recognizing and binding the NT-3 protein; and

a siRNA nucleic acid (small interfering RNA) capable of inhibiting the expression of NT3 in cells, preferably in vivo,

as a medicament.

The amino acid sequence of mammal, such as human, neurotrophin 3 or TrkC receptor are well known by the skilled man. Example of these amino acid sequences with the localization of their particular domain can be found in Genbank under the accession number AAA59953 (dated Jan. 5, 1995) for human neurotrophin 3 or AAB33111 for human TrkC. Preferably, in the compounds of the present invention, said extracellular domain of TrkC receptor or fragment thereof comprises the first 300 N-terminal amino acid residues, preferably 350, 375, 400, 410, 420 and 429 amino acid residues. More preferably NT3 and TrkC are from mammal such as from mouse, rat or human.

In another aspect, the present invention pertains to the use of the level of neurotrophin 3 expression as a marker for the identification of metastatic cancer in a patient, preferably of metastatic neuroblastoma or metastatic breast cancer. In another aspect, the present invention pertains to a method of treatment for inducing the apoptosis or the cell death of tumor cells which have acquired the selective advantage to escape TrkC dependence receptors induced apoptosis, preferably by elevated neurotrophin 3 level, in a patient comprising administering a compound able to inhibit the interaction between neurotrophin 3 and its TrkC receptor, a compound able to inhibit the dimerization or the multimerization of the TrkC receptor, a compound according to the present invention, or selected by the method of the present invention, in said patient in need thereof. In another aspect, the present invention pertains to a method for the prevention or for the treatment of cancer in a patient comprising administering a compound according to the present invention, or selected by the method of the present invention, in said patient in need thereof.

The present invention also comprises the use of a compound according to the present invention, or selected by the method of the present invention, for the manufacture of a medicament for the prevention or the treatment of cancer in mammals, including man. Preferably said cancer is a metastatic or an aggressive cancer. More preferably, in the method of treatment or in the use of a compound according to the present invention, said cancer is selected from the group consisting of neuroblastoma and breast cancer. More preferably, in the method of treatment or in the use of a compound according to the present invention, the primary tumor cells of said cancer express or overexpress neurotrophin 3. The term “antibody” as used herein refers to immunoglobulin molecules and immunologically active portions of immunoglobulin molecules, i.e., molecules that contain an antigen binding site which specifically binds (immunoreacts with) the neurotrophin 3 protein or its receptor.

Preferably, the antibody is TrkC specific and does not recognize TrkA or B receptor. Evidence of the specificity of antibody for TrkC and its lack of cross-reactivity with the other Trk family members can be provided by immunocytochemical analysis of mammals recombinant cells such as HEK 293 cells expressing TrkA, B, or C or by immunoblots. The term “antibody” comprises monoclonal or polyclonal antibodies but also chimeric or humanized antibodies.

An isolated neurotrophin 3 protein or TrkC receptor protein, or a specific fragment thereof can be used as an immunogen to generate antibodies that bind such protein using standard techniques for polyclonal and monoclonal antibody preparation. It may be also possible to use any fragment of these protein which contains at least one antigenic determinant may be used to generate these specific antibodies. A protein immunogen typically is used to prepare antibodies by immunizing a suitable subject, (e.g., rabbit, goat, mouse or other mammal) with the immunogen. An appropriate immunogenic preparation can contain said protein, or fragment thereof, and further can include an adjuvant, such as Freund's complete or incomplete adjuvant, or similar immunostimulatory agent. Thus, antibody for use in accordance with the invention include either polyclonal, monoclonal chimeric or humanized antibodies. antibodies able to selectively bind, or which selectively bind to an epitope-containing a polypeptide comprising a contiguous span of at least 8 to 10 amino acids of an amino acid sequence of the neurotrophin 3 protein or its TrkC receptor.

A preferred agent for detecting and quantifying mRNA or cDNA encoding neurotrophin 3 protein, is a labeled nucleic acid probe or primers able to hybridize this mRNA or cDNA. The nucleic acid probe can be an oligonucleotide of at least 10, 15, 30, 50 or 100 nucleotides in length and sufficient to specifically hybridize under stringent conditions to the mRNA or cDNA. The nucleic acid primer can be an oligonucleotide of at least 10, 15 or 20 nucleotides in length and sufficient to specifically hybridize under stringent conditions to the mRNA or cDNA, or complementary sequence thereof.

A preferred agent for detecting and quantifying the neurotrophin 3 protein, is an antibody able to bind specifically to this protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. For example, in vitro techniques for detection of candidate mRNA include Northern hybridizations and in situ hybridizations. In vitro techniques for detection of the candidate protein include enzyme linked immunosorbent assays (ELISAs), Western blots, immunoprecipitations and immunofluorescence. In vitro techniques for detection of candidate cDNA include Southern hybridizations.

When the invention encompasses kits for quantifying the level of neurotrophin 3 protein, the kit can comprise a labeled compound or agent capable of quantifying these proteins. Said agents can be packaged in a suitable container. The kit can further comprise instructions for using the kit to quantify the level of the neurotrophin 3 protein or of the neurotrophin 3 transcript.

In certain embodiments of the method of the present invention, the determination of the neurotrophin 3 transcripts involves the use of a probe/primer in a polymerase chain reaction (PCR), such as anchor PCR or RACE PCR, or, alternatively, in a ligation chain reaction (LCR) (see, e.g., Landegran et al., 1988, Science 241:23-1080; and Nakazawa et al., 1994, Proc. Natl. Acad. Sci. USA, 91:360-364), or alternatively quantitative real time RT-PCR This method can include the steps of collecting a sample of cells from a patient, isolating nucleic acid (e.g. mRNA) from the cells of the sample, optionally transforming mRNA into corresponding cDNA, contacting the nucleic acid sample with one or more primers which specifically hybridize to the neurotrophin 3 or mRNA or their corresponding cDNA under conditions such that hybridization and amplification of the neurotrophin 3 mRNA or cDNA occurs, and quantifying the presence of the amplification products. It is anticipated that PCR and/or LCR may be desirable to use as an amplification step in conjunction with any of the techniques used for quantifying nucleic acid detecting.

The methods described herein may be performed, for example, by utilizing pre-packaged diagnostic kits comprising at least one probe nucleic acid or set of primer or antibody reagent described herein, which may be conveniently used, e.g., in clinical settings to follow-up or diagnose patients.

Finally, the present invention is related to the use of antisense or iRNA (interfering RNA) oligonucleotides specific of the nucleic acid encoding neurotrophin 3 protein for the manufacture of a medicament intended to prevent or to treat metastatic or aggressive cancer, preferably said cancer is selected from the group consisting of cancer related to overexpression of NT3, preferably neuroblastoma. Interfering RNA (iRNA) is a phenomenon in which a double stranded RNA (dsRNA) specifically suppresses the expression of a gene bearing its complementary sequence. iRNA has since become a useful research tool for many organisms. Although the mechanism by which dsRNA suppresses gene expression is not entirely understood, experimental data provide important insights. This technology has great potential as a tool to study gene function in mammalian cells and may lead to the development of pharmacological agents based upon siRNA (small interfering RNA).

When administered to a patient, a compound of the present invention is preferably administered as component of a composition that optionally comprises a pharmaceutically acceptable vehicle. The composition can be administered orally, or by any other convenient route, and may be administered together with another biologically active agent. Administration can be systemic or local. Various delivery systems are known, e.g., encapsulation in liposomes, microparticles, microcapsules, capsules, etc., and can be used to administer the selected compound of the present invention or pharmaceutically acceptable salts thereof.

Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically. The mode of administration is left to the discretion of the practitioner. In most instances, administration will result in the release of the compound into the bloodstream or directly in the primary tumor.

Compositions comprising the compound according to the invention or selected by the methods according to the present invention, form also part of the present invention. These compositions can additionally comprise a suitable amount of a pharmaceutically acceptable vehicle so as to provide the form for proper administration to the patient. The term “pharmaceutically acceptable” means approved by a regulatory agency or listed by a national or a recognized pharmacopeia for use in animals, mammals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gelatin, starch and the like. In addition, auxiliary, stabilizing, thickening, lubricating and coloring agents may be used. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, sodium stearate, glycerol monostearate, sodium chloride, dried skim milk, glycerol, propylene, glycol, water and the like. Test compound compositions, if desired, can also contain minor amounts of wetting or emulsifying agents, or pH buffering agents. The compositions of the invention can take the form of solutions, suspensions, emulsion, tablets, pills, pellets, capsules, capsules containing liquids, powders, sustained-release formulations, suppositories, emulsions, aerosols, sprays, suspensions, or any other form suitable for use. Said composition is generally formulated in accordance with routine procedures as a pharmaceutical composition adapted to human beings for oral administration or for intravenous administration. The amount of the active compound that will be effective in the treatment can be determined by standard clinical techniques. In addition, in vitro or in vivo assays may optionally be employed to help identify optimal dosage ranges. The precise dose to be employed will also depend on the route of administration, and the seriousness of the disease, and should be decided according to the judgment of the practitioner and each patient's circumstances. However, suitable dosage ranges for oral, intranasal, intradermal or intraveneous administration are generally about 0.01 milligram to about 75 milligrams per kilogram body weight per day, more preferably about 0.5 milligram to 5 milligrams per kilogram body weight per day.

It is to be understood that while the invention has been described in conjunction with the above embodiments, that the foregoing description and the following examples are intended to illustrate and not limit the scope of the invention. Other aspects, advantages and modifications within the scope of the invention will be apparent to those skilled in the art to which the invention pertains.

### BRIEF DESCRIPTION OF THE DRAWINGS

FIGS. 1A-1G:

TrkC is a dependence receptor. HEK293T (A-C) or 13.S.24 (D-G) cells were transfected with the mock plasmid (Mock) or expression plasmid TrkA, TrkB, or TrkC. (A) Cell death induction by TrkC measured by trypan blue exclusion. Standard deviations are indicated (n=3). (B) TrkC induced increased caspase activity, monitored by cleavage of the Ac-DEVD-AFC substrate. zVAD-fmk, a general and potent caspase inhibitor, was also used. (C) TrkC induces caspase-3 activation as measured by immunostaining with antiactive caspase-3 antibody. Representative images are shown. Quantification is indicated together with standard deviations (n=3). (D) Transfected cells were labeled with FITC-VAD-fmk. As a control, a kinase-inactive (see FIG. 4C) TrkC (TrkC D679N) was also transfected. A representative flow cytometry analysis is shown. Note that TrkC induces caspase activation, whereas TrkA and TrkB behave like mock transfection. (E-G) Mock 13.5.24 cells or rat (E and F)/human (G) TrkC transfected 13.S.24 cells were treated with increasing doses of NT-3 (0.5, 1, 2.5, 5, 10, and 50 ng/ml as indicated for rat TrkC and 10 ng/ml for human TrkC). (E) Cell death induction by TrkC measured by trypan blue exclusion (Upper). Phosphorylation of Akt and Erk is shown by immunoblot with anti-phospho Akt and anti-phospho Erk (Lower). A loading control is indicated by immunoblot on total Erk. (F) NT-3 inhibits TrkC-induced caspase activity, as monitored by using the Ac-DEVD-AFC substrate. Note that the level of TrkC expression is similar in the different tested conditions as shown by Western blot. (G) Human TrkC instead of rat TrkC was expressed in 13.S.24 cells. Cell death was measured by enumerating the cells labeled with active caspase-3. A ratio is presented as active caspase-3-positive cells in each condition to the one detected in the mock transfection. Standard deviations are indicated (n=3).

FIGS. 2A-2D:

TrkC is a caspase substrate. (A) TrkC is mainly cleaved by caspase-3 in vitro. The in vitro-translated intracellular domain of TrkC, but also of TrkA and TrkB, was incubated in the absence of caspase or with purified caspase-3 or caspase-8 (0.3 μM). An autoradiograph is shown. (B) Aspartic acid residues 641 and 495 are the cleavage sites. As shown in the lower autoradiograph, the TrkC IC D495/D641N mutant is not cleaved by caspase-3. (C) TrkC wild type or mutated at either single (TrkC D495N, TrkC D641N) or both (TrkC D495N/D641N) cleavage sites were expressed in 13.S.24 cells in the presence or absence of z-VAD-fmk. (D) Semidissociated DRG were left untreated (−) or treated overnight with NT-3 or with the TrkC blocking antibody AF1404 together with BAF or not. In C and D, cleavage fragments are observed by Western blot with a TrkC C terminus-directed antibody. D Top shows the full-length TrkC, whereas D Middle shows a 20-kDa fragment. D Bottom is a loading control revealing actin.

FIGS. 3A-3I:

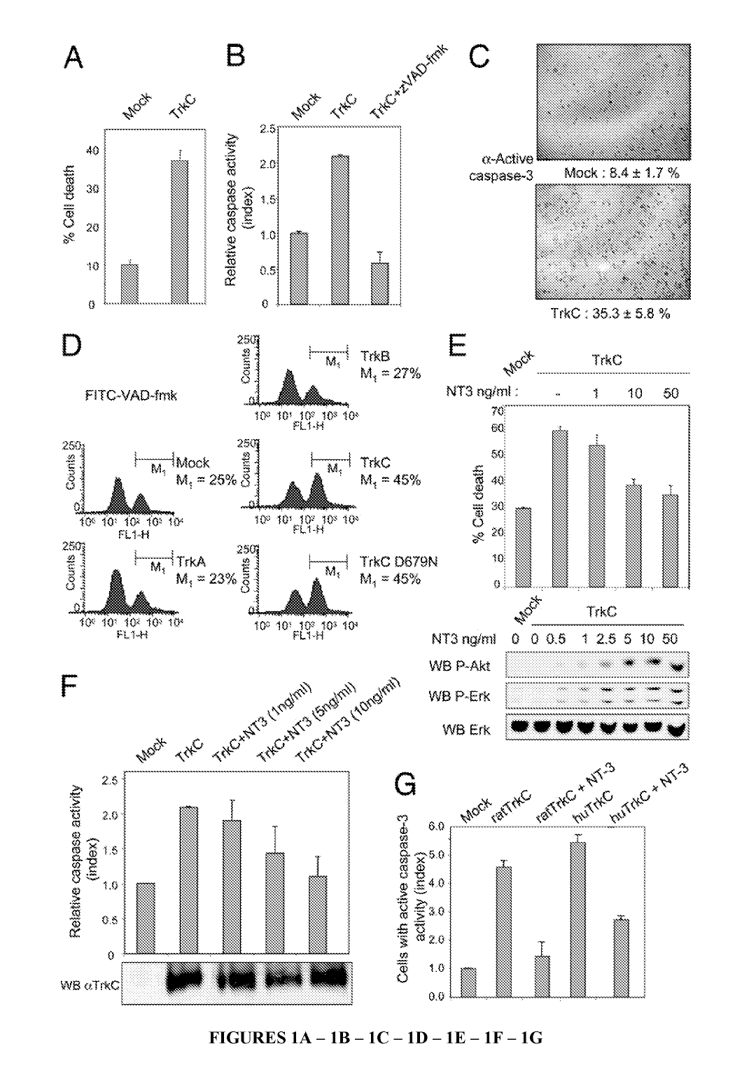

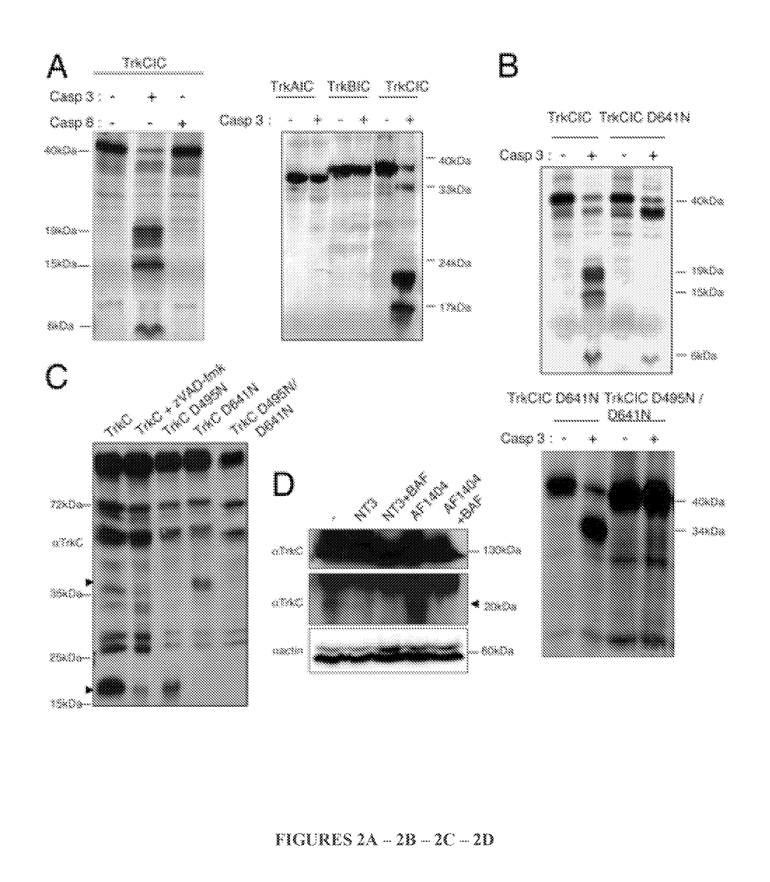

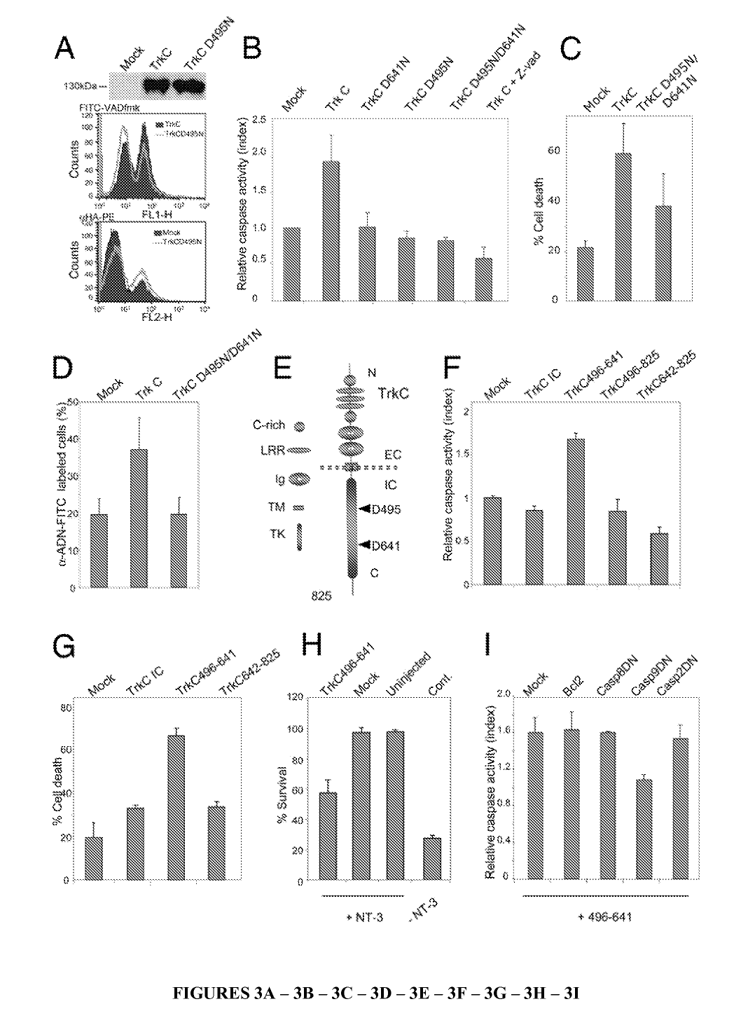

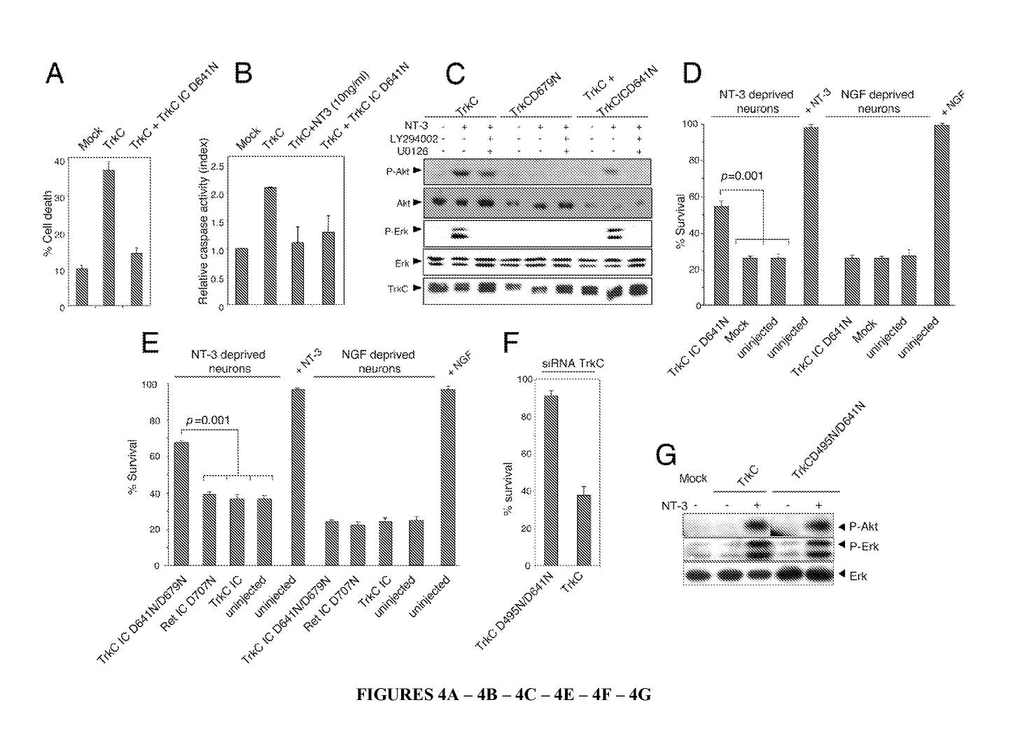

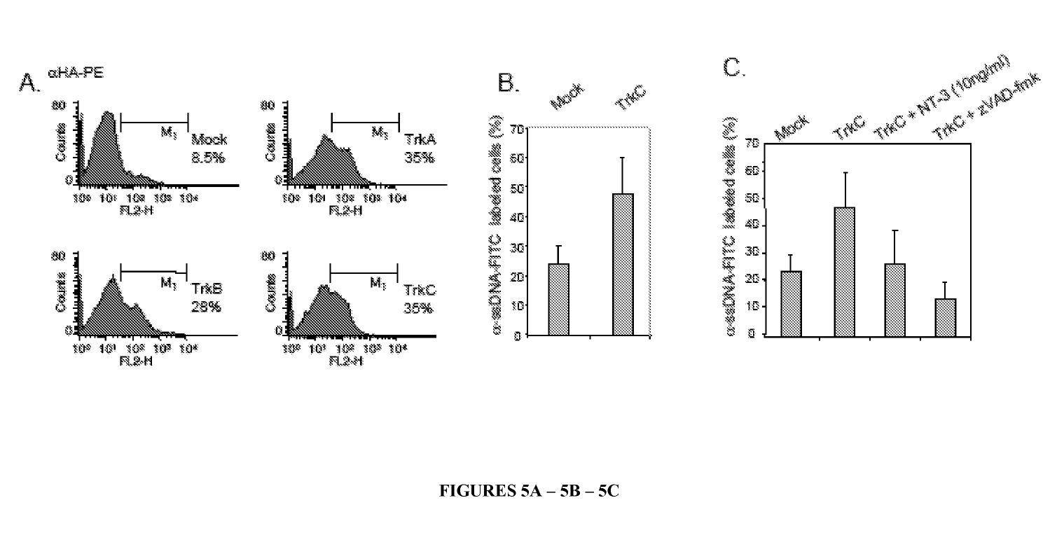

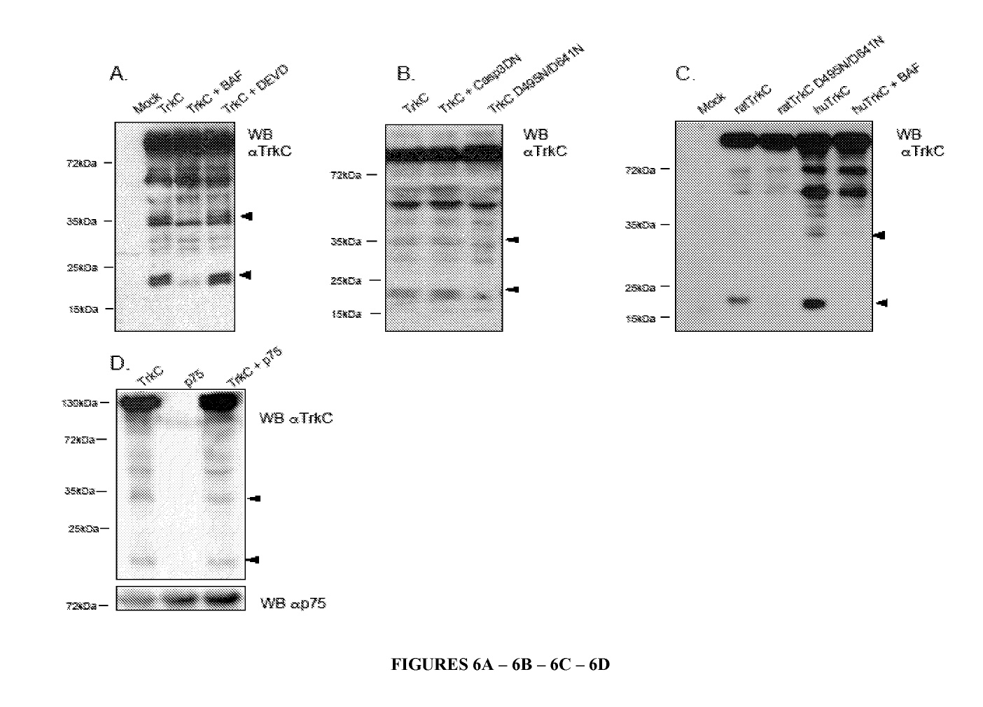

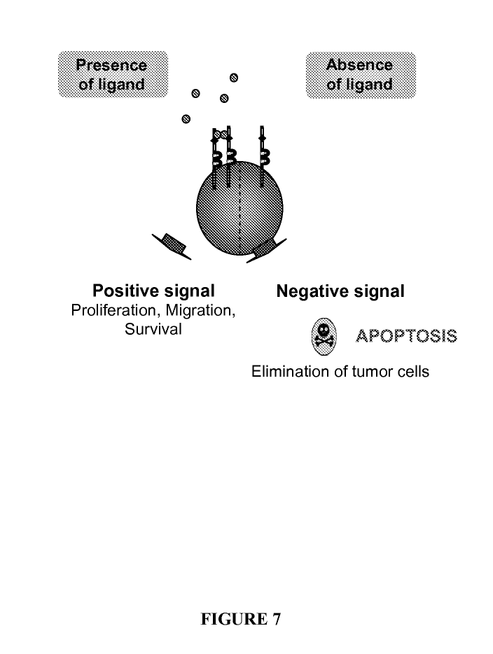

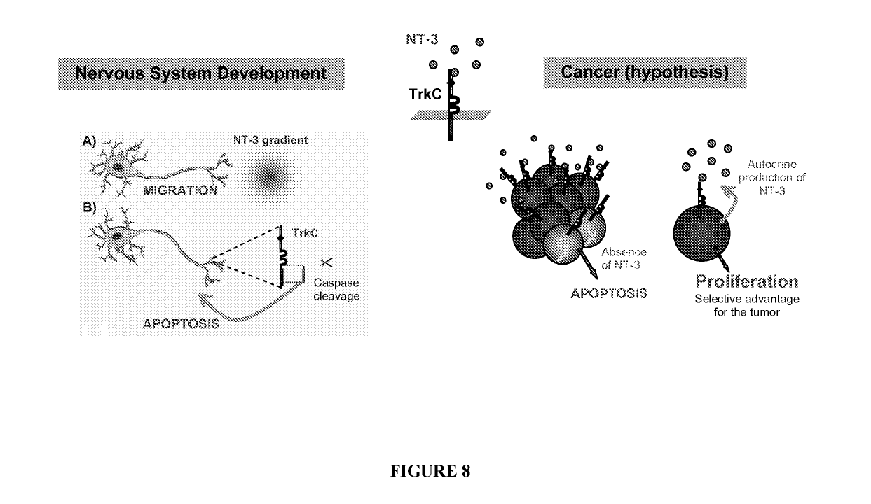

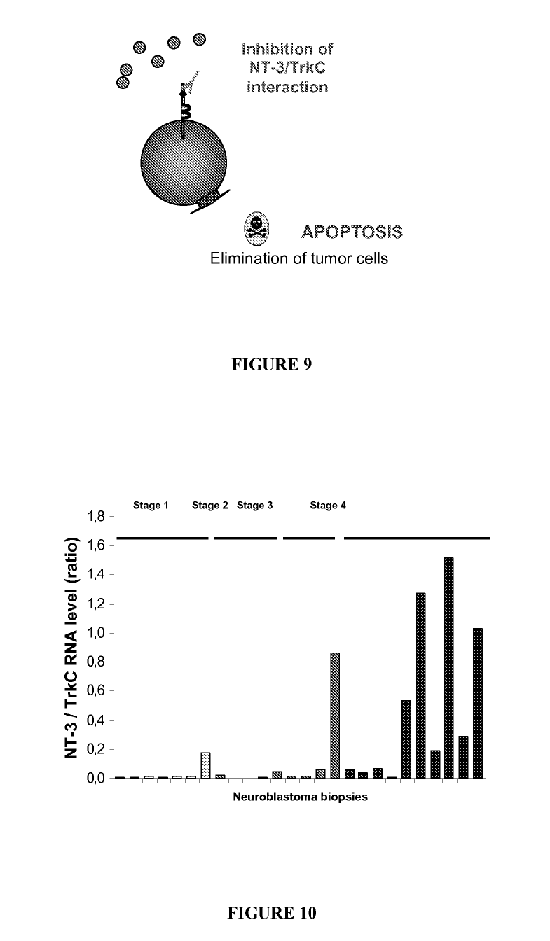

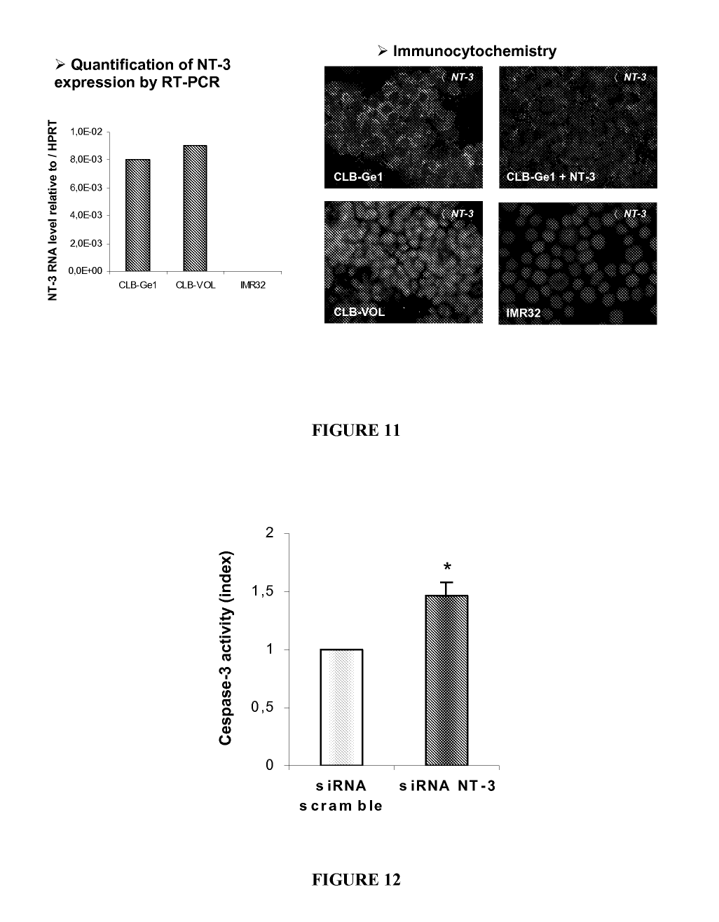

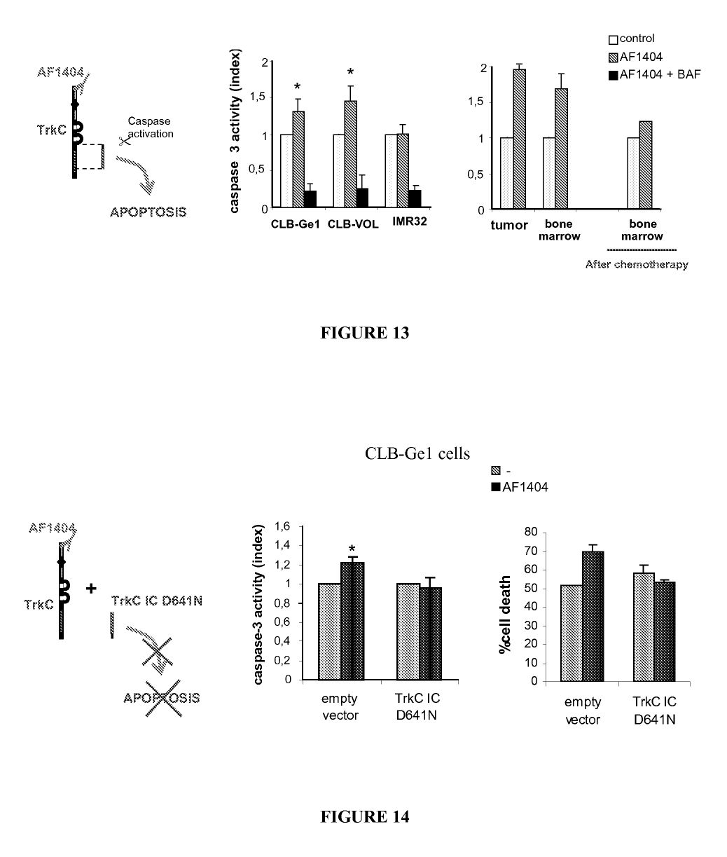

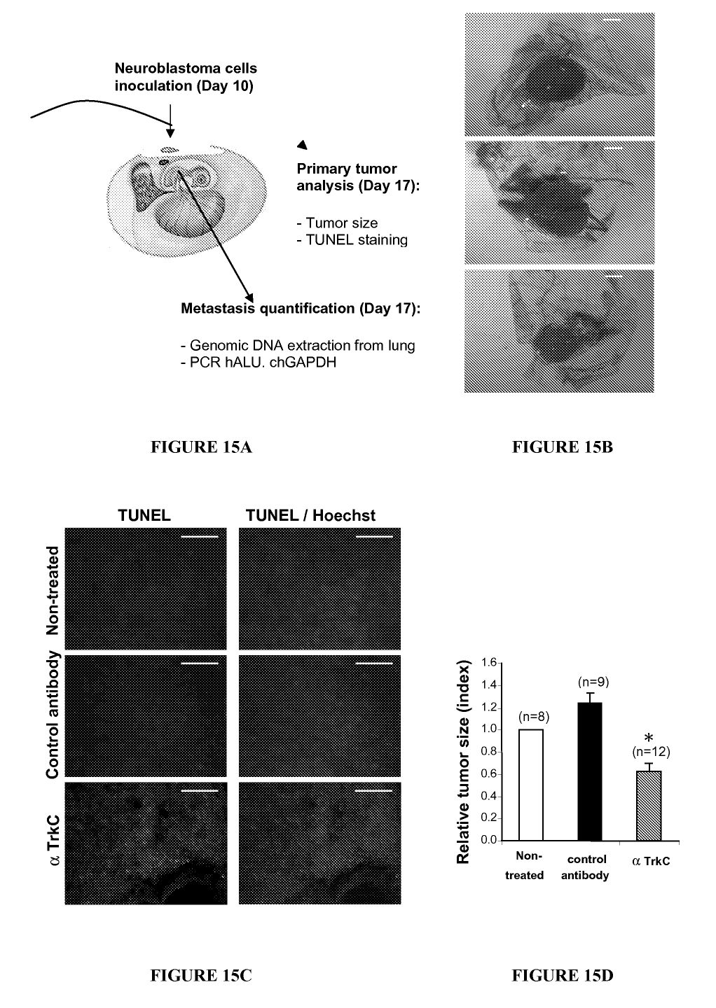

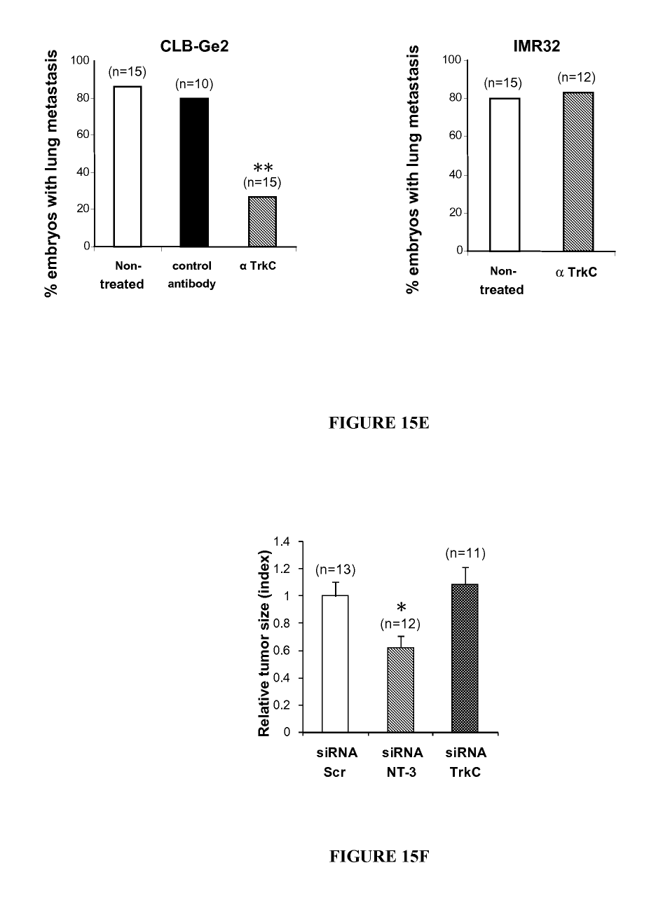

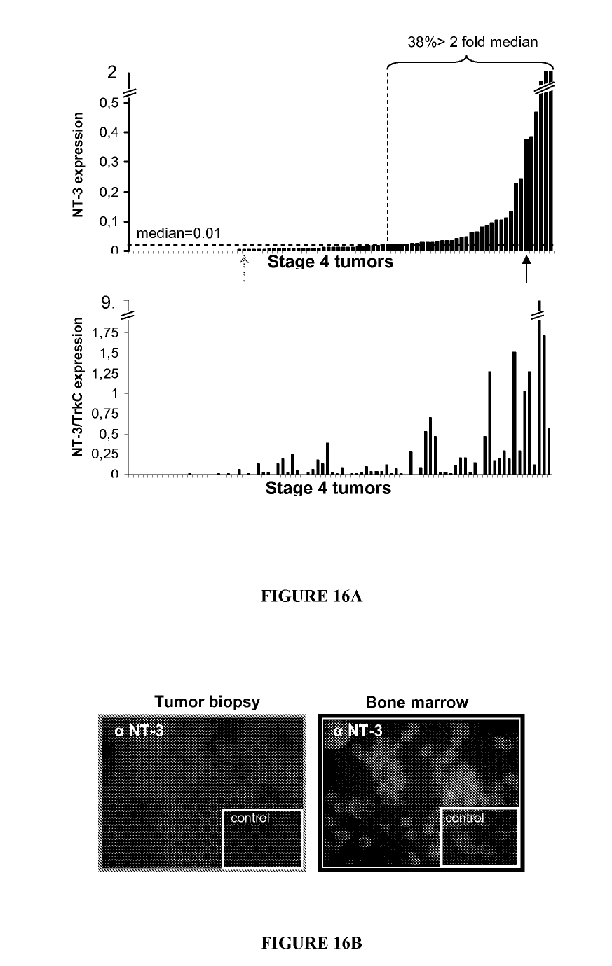

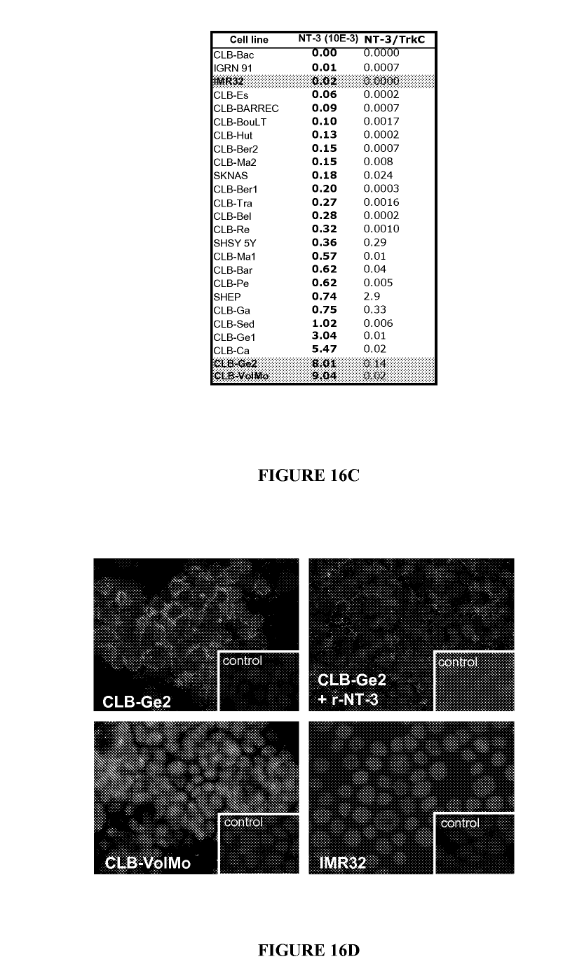

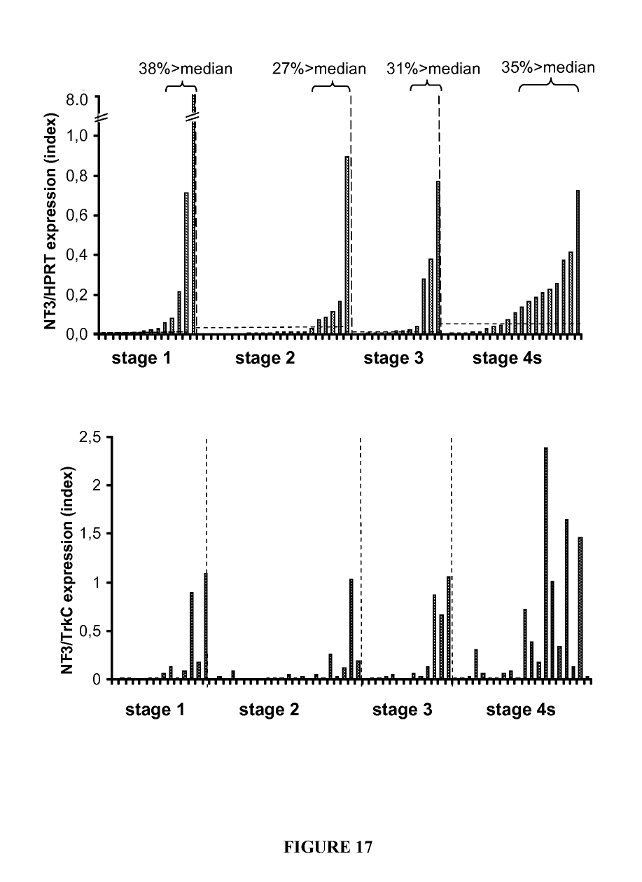

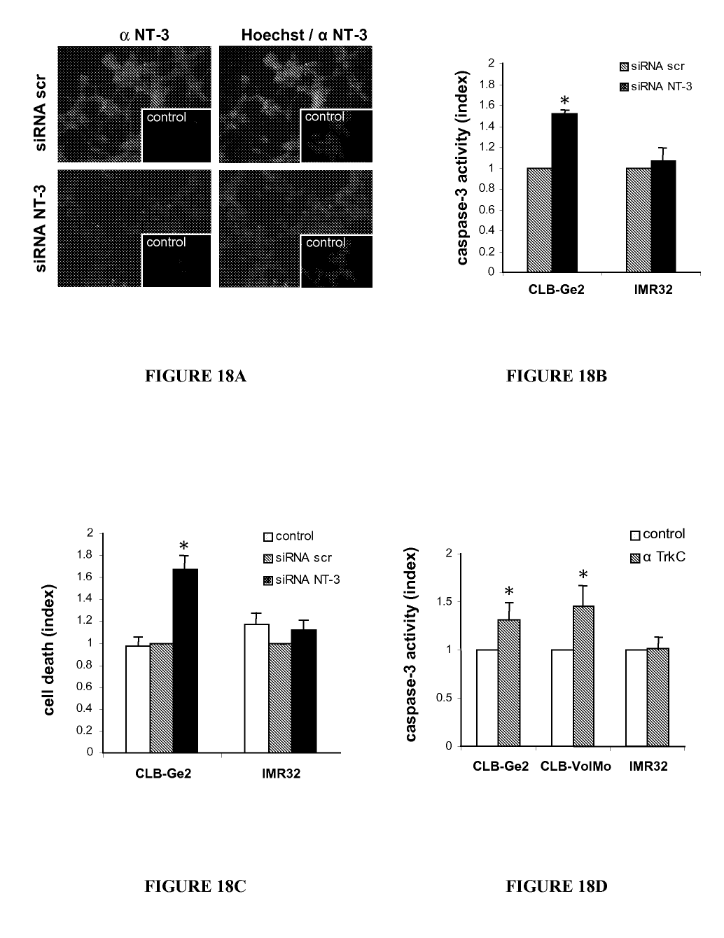

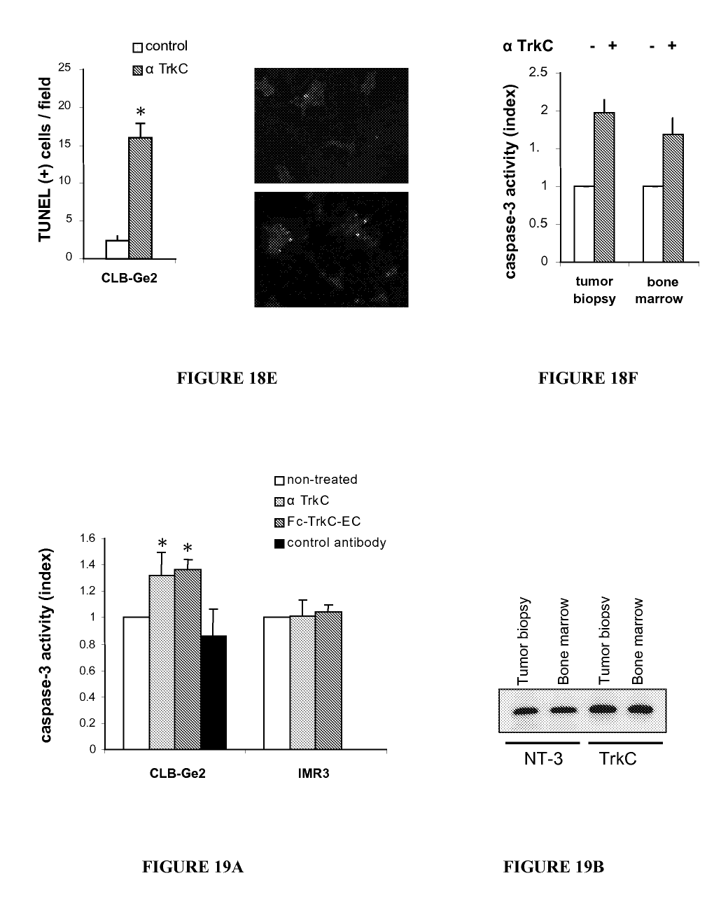

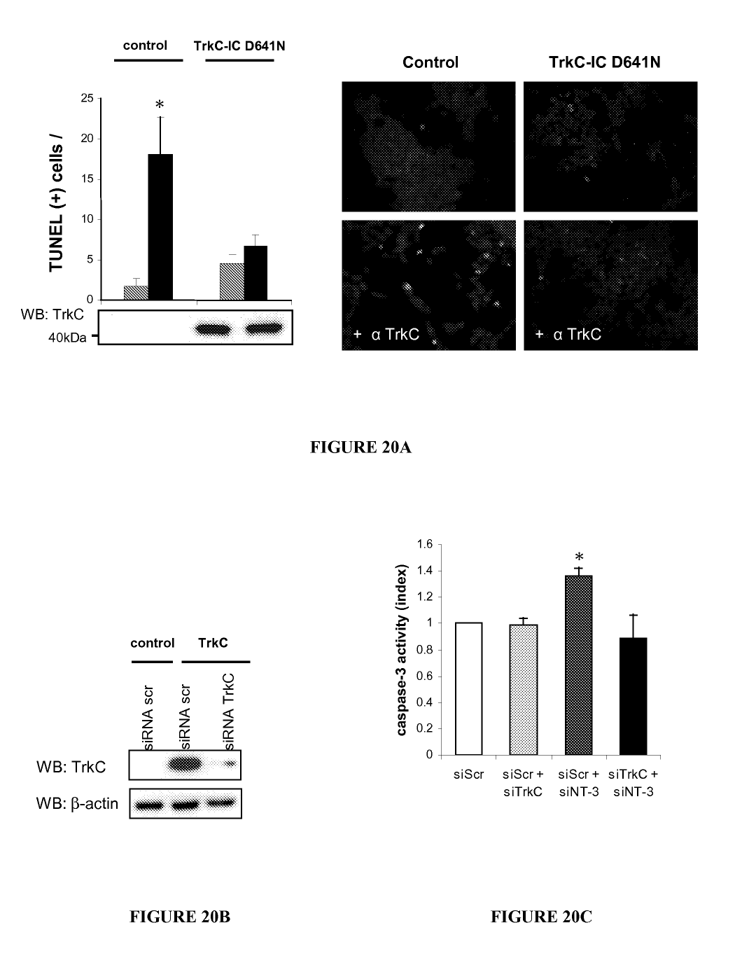

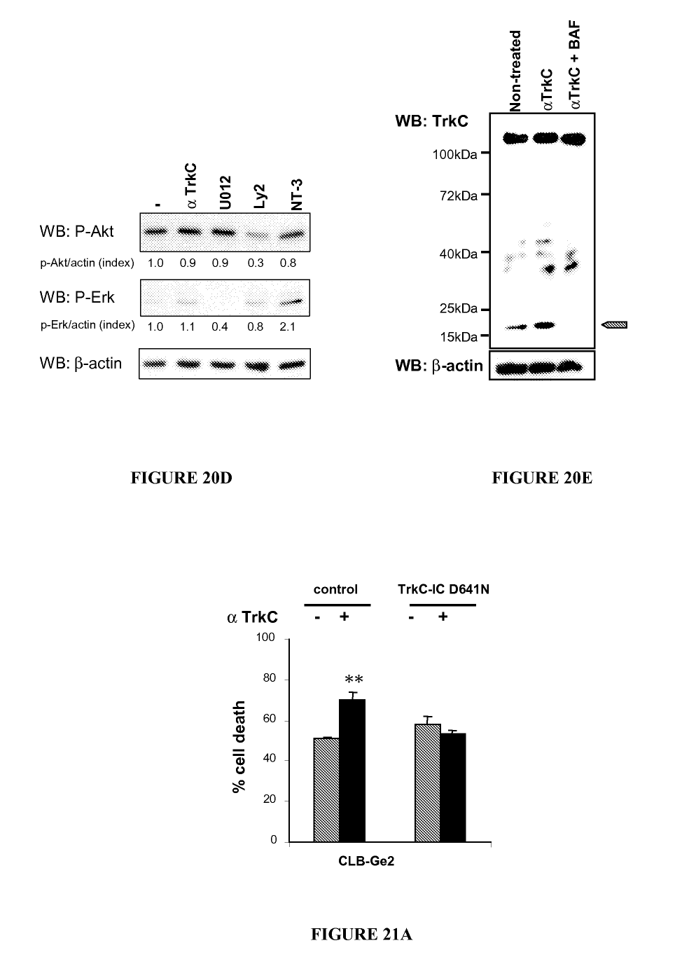

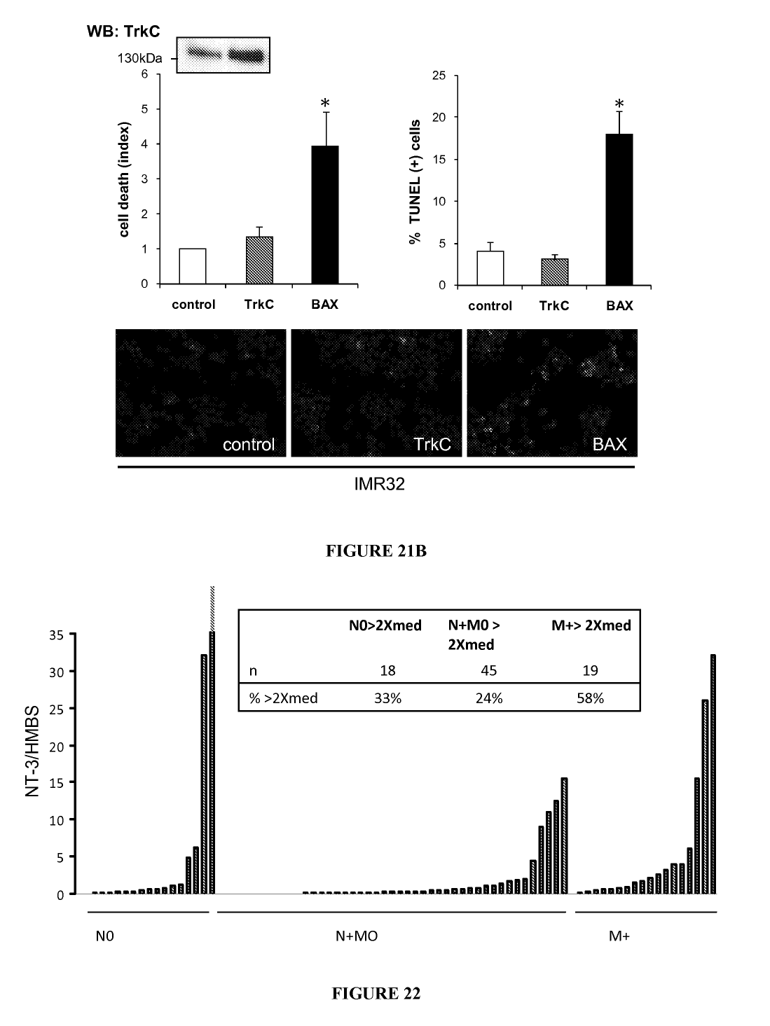

TrkC cleavage releases a proapoptotic domain. (A-D) Mutation of one or two caspase cleavage sites of TrkC inhibits the proapoptotic activity of TrkC. (A) Mock plasmid- (Mock), TrkC-, or TrkC D495N-transfected 13.5.24 cells were analyzed by Western blot with anti-HA (HA-TrkC) antibody, by FACS analysis for membrane localization, as in SI FIG. 5A, and by FACS analysis for measurement of caspase activity, as in FIG. 1D. (B) Single or double caspase-site mutants were transfected into HEK293T cells, and caspase activity was measured by DEVD-AFC cleavage in cell lysates, as in FIG. 1B. (C and D) TrkC D495N/D641N mutant is not proapoptotic in HEK293T cells, as measured by trypan blue exclusion (C) or by DNA condensation as in SI FIG. 5B (D). Standard deviations are indicated (n=3). (E) TrkC is composed of 825 amino acids. Caspase cleavage sites are located at D495 and D641 in the cytoplasmic region of the receptor. EC, extracellular domain; IC, intracellular domain; TK, tyrosine kinase domain; LRR, leucin-rich repeat; Ig, Ig domain; C-rich: cystein-rich domain; TM, transmembrane domain. (F and G) The fragment released by caspase cleavage (496-641) is a potent cell death inducer when expressed in 13.S.24 neuroblasts, as measured by a caspase activity assay based on FITC-VAD-fmk, as in FIG. 1D (F), or by trypan blue exclusion (G). Standard deviations are indicated (n=3). (H) Primary sensory neurons were maintained with NT-3, microinjected with a mock plasmid (Mock) or with the plasmid encoding TrkC 496-641. Living neurons were counted 72 h later and expressed as the percentage of initially injected neurons. The standard errors of the means are shown (n=3). (I) 13.5.24 cells were cotransfected with expression plasmids for TrkC 496-641 (or empty vector) and either empty vector (Mock), Bcl-2, or dominant-negative (DN) caspase-2, -8, or -9. Apoptosis was measured by monitoring caspase activity as in FIG. 1D. Caspase activity is presented as the ratio between the TrkC 496-641-transfected population and the control-transfected population for each cotransfection (Mock, Bcl2, Casp2DN, Casp8DN, and Casp9DN). Standard deviations are indicated (n=3).

FIGS. 4A-4G:

DRG neurons death upon NT-3 loss depends on the proapoptotic activity of TrkC. (A and B) TrkC IC D641N acts as a dominant-negative mutant of TrkC. HEK293T cells were transfected with the mock plasmid (Mock), the TrkC expression plasmid together with the mock plasmid, or the TrkC IC D641N expression plasmid. The dominant-negative effect of TrkC IC D641N was measured by trypan blue exclusion (A) or a caspase activity assay, as in FIG. 1B (B). Standard deviations are indicated (n=3). (C) 13.S.24 neuroblasts were transfected with TrkC wild type or TrkC kinase-dead (TrkCD679N), or cotransfected with TrkC and the dominant-negative mutant TrkC IC D641N, in the presence or absence of 10 ng/ml NT-3 and with or without the addition of a PI3K inhibitor (LY294002, 10 μM) or a MEK inhibitor (U0126, 10 μM). Akt and Erk phosphorylation was visualized by Western blot with an anti-phosphoAkt and an anti-phosphoErk antibody, respectively. The levels of Akt and Erk kinases were shown by reprobing the membrane with an anti-total Akt antibody or an anti-total Erk antibody, respectively. TrkC immunoblot is also shown. Similar results were obtained by using the FACE-Akt ELISA (Active Motif, Carlsbad, Calif.). (D and E) Sensory neurons were maintained with NT-3 or NGF, microinjected with a mock plasmid (Mock) or the plasmids encoding TrkC IC D641N (D) or kinase dead TrkC IC D641N/D679N, the dominant-negative mutant of Ret (i.e., Ret IC D707N) or TrkC IC (E), and grown further without NT-3 or NGF. Living neurons were counted 72 h later and expressed as the percentage of initially injected neurons. Experiments shown in D and E were performed separately. (F) Same as D and E: sensory neurons were maintained with NT-3, microinjected with endogenous TrkC siRNA and either TrkC or TrkC D495N/D641N, and grown further in the absence of NT-3. Living neurons were counted 72 h later and expressed as the percentage of initially injected neurons. The standard errors of the means are shown (n=3). (G) Same as in C, except that Akt/Erk phosphorylation was measured in 13.S.24 cells transfected with TrkC or TrkC D495N/D641N.

FIGS. 5A-5C:

TrkC behaves as a dependence receptor. 13.S.24 (A and C) or HEK293T (B) cells were transfected with the mock (Mock), TrkA, TrkB, or TrkC expression plasmid. (A) Membrane localization of HA-tagged TrkA, TrkB, and TrkC was monitored by FACS analysis using anti-HA antibody (aHA-PE). (B) Transfected cells were labeled with an anti-single-stranded DNA antibody, after incubation of the cells in formamide at 75° C. DNA in apoptotic cells is denatured at 75° C. and analyzed by flow cytometry. The percentage of cells stained with the FITC-antibody is shown and internal standard deviations are indicated. (C) NT-3 inhibits TrkC-induced apoptosis. 13.S.24 cells were mock- or TrkC-transfected and NT-3 (10 ng/ml) or zVAD-fmk were added after 3 and 24 h of transfection. NT-3 inhibits TrkC induced apoptosis, as monitored by labeling cells with the anti-single-stranded DNA antibody as in B.

FIGS. 6A-6D:

TrkC cleavage is not dependent on caspase-3 or p75 ntr and is also occurring in human TrkC. (A and B) TrkC was transfected in 13.S.24 cells, in the presence or the absence of either the general caspase inhibitor BAF or the specific caspase-3 inhibitor DEVD-fmk (A), or cotransfected with caspase-3 dominant-negative (Casp3DN) (B). As a control, TrkCD495N/D641N was also transfected to indicate the cleavage bands that are disappearing in this mutant. (C) Human or rat TrkC expressing constructs were transfected in 13.S.24 cells. Cleavage fragments are observed using the C terminus-directed antibody that recognizes human as well as rat TrkC and are indicated with arrows. As shown for rat TrkC (A), human TrkC is no longer cleaved in the presence of the general caspase inhibitor BAF. (D) 13.S.24 cells were transfected with TrkC or cotransfected with TrkC and p75 ntr, and TrkC cleavage was analyzed as in C. Note that the presence of an elevated level of p75 ntr detected by p75 ntr immunoblot has no significant effect on the TrkC cleavage. Moreover, TrkC transfection in Daoy cells that fail to express p75 ntr is associated with appearance of similar caspase-dependent cleavage of TrkC (data not shown).

FIG. 7: Scheme showing the Dependence Receptor concept.

FIG. 8: Scheme showing TrkC as a Dependence Receptor.

FIG. 9: Scheme showing TrkC as a target for Neuroblatoma (NB).

FIG. 10: NT-3 and TrkC RNA from 26 NB tumor samples was measured by quantitative PCR.

FIG. 11: Screening of human tumor cell lines: Quantification of NT-3 expression by RT-PCR.

Several NB cell lines were screened by measuring NT-3 and TrkC expression. CLB-Ge1 and CLB-Vol.Mo cell lines were selected by their high NT-3 expression, while IMR32 cells were chosen as a negative control cell line. Confirmation by immunochemistry.

FIG. 12: NT-3 siRNA induces apoptosis of CLB-Ge1 cells.

FIG. 13: Interference of NT-3 interaction with TrkC induces apoptosis of NT-3 high NB cells: NT-3 blocking antibody (AF1404) induces apoptosis in NT-3 expressing NB cell lines and stage 4 patient samples.

FIG. 14: Interference of NT-3 interaction with TrkC triggers apoptosis through TrkC. Transfection of TrkC dominant negative (TrkC IC D641N) blocks AF1404-induced apoptosis.

FIGS. 15A-15F: The inhibition of NT-3/TrkC interaction reduces tumor progression and metastasis of NT-3 expressing NB cells in a chick model; Blocking NT-3/TrkC inhibits NB growth and dissemination.

(A) Schematic representation of the experimental chick model used in B-E. IMR32 or CLB-Ge2 cells were grafted in CAM at day 10 and α TrkC antibody or an isotypic antibody (control antibody) was added on day 11 and day 14. Tumors and lungs were harvested on day 17.

(B-C-D) Effect of α TrkC antibody on primary tumor growth and apoptosis.

(B) Representative images of CLB-Ge2 primary tumors formed on non-treated CAM or treated either with an isotypic antibody (control antibody) or with α TrkC antibody. Scale bars correspond to 2 mm.

(C) Representative images of TUNEL staining in the respective primary tumors described in B. Scale bars correspond to 100 μm.

(D) Quantitative analysis showing the primary tumor size relative to non-treated tumors.

(E) Effect of α TrkC antibody on lung metastasis. Percentage of embryos with lungs invaded by IMR32 or CLB-Ge2 cells after two intratumoral injections (day 11 and day 14) of either α TrkC antibody, an isotypic antibody or non-treated.

(F) Effect of NT-3 siRNA and TrkC siRNA on primary tumors. CLB-Ge2 cells were grafted in CAM at day 10 and NT-3, TrkC or scramble siRNA were injected intraveneously on a chorioallantoic vessel on day 11 and day 14. Primary tumors were harvested on day 17. In D and F, errors bars indicate s.e.m; * indicates a p<0.05 calculated by a two-sided Mann-Whitney test compared to non-treated tumors. In E, * indicates a p<0.01 calculated by a Chi square test.

FIGS. 16A-16D: NT-3 is expressed in a large fraction of stage 4 NB.

(A) NT-3 expression and NT-3/TrkC ratio measured by Q-RT-PCR on total RNA from tumors from a total of 86 stage 4 NB patients (20 stage 4S NB are shown in FIG. 17). The percentage of tumors expressing NT-3 more than two fold of the value corresponding to the median is indicated.

(B) Representative NT-3 immunohistochemistry on a tumor biopsy and bone marrow dissociated cells from low (left panel) and high (right panel) NT-3-expressing stage 4 patients, which corresponds to dotted-gray arrow and black arrow on FIG. 1A respectively. Inset: a control without antibody is presented.

(C) NT-3 expression and NT-3/TrkC ratio measured by Q-RT-PCR in a fraction of NB cell lines. HPRT expression was used as an internal control.

(D) Representative NT-3 immunohistochemistry on CLB-Ge2, CLB-VolMo, and IMR32 cells. Inset: Control without primary antibody. Upper right panel, NT-3 immunostaining when an excess of recombinant NT-3 (r-NT-3) is added with primary antibody. Note that the two upper panels show immunohistochemistry performed in absence of membrane permeabilization (without Triton X-100) while the two below panels were performed after cell permeabilization (with Triton X-100).

FIG. 17: NT-3 is expressed in a large fraction of stage 1, 2, 3, 4s NB.

NT-3 and TrkC expression measured by Q-RT-PCR on total RNA from tumors from a total of 69 NB patients (14 stage 1, 22 stage 2, 13 stage 3 and 20 stage 4s). The percentage of tumors expressing NT-3 more than two fold of the value corresponding to the median is indicated (upper panel). HPRT expression was used as an internal control. NT-3/TrkC ratio is described in the lower panel.

FIGS. 18A-18F: Disruption of NT-3 autocrine loop triggers NB cell death.

(A) NT-3 Immunostaining on CLB-Ge2 cell line 24 h after transfection with scramble siRNA (siRNA scr.) or with NT-3 siRNA (siRNA NT-3). Inset: Control without primary antibody.

(B-C) Cell death induction in IMR32 and CLB-Ge2 cell lines was quantified in non transfected cells (control) or after transfection with either scramble siRNA (siRNA scr) or NT-3 siRNA (siRNA NT-3), using relative caspase-3 activity assay (B), or Toxilight assay (C).

(D-E) Cell death induction in CLB-Ge2, CLB-VolMo or IMR32 cell lines was quantified in cells treated with (TrkC) or without (control) anti-TrkC antibody, using relative caspase-3 activity assay (D), or TUNEL assay (E). For the TUNEL assay, representative fields of TUNEL staining are shown (upper panel: control cells, lower panel: cells treated with TrkC (“TrkC” for anti-TrkC antibody).

(F) Effect of TrkC antibody on stage 4 NB tumoral cells directly dissociated from the surgical biopsy and plated for 24 h in presence (+) or in absence (−) of treatment. In B-F, error bars indicate s.e.m.; * indicates a p<0.05 calculated by using a two-sided Mann-Whitney test, compared to control.

FIGS. 19A-19B: NT-3/TrkC interference promotes neuroblastoma cell death.

(A) Cell death induction in CLB-Ge2 or IMR32 cell lines was quantified in cells treated either with (α TrkC) anti-TrkC antibody, TrkC extracellular domain (Fc-TrkC-EC) or with an isotypic control (control antibody) by relative caspase-3 activity assay. Error bars indicate s.e.m.; * indicates a p<0.05 calculated by using a two-sided Mann-Whitney test, compared to control.

(B) NT-3 and TrkC expression was amplified by RT-PCR on cDNA extracted from the tumor biopsy and the bone marrow taken from a stage 4 NB patient, and visualized on agarose gel.

FIGS. 20A-20E: NT-3/TrkC interference promotes TrkC pro-apoptotic activity.

(A) CLB-Ge2 cells were transfected with either empty vector (control) or with a plasmid encoding the dominant negative TrkC-IC D641N and treated 24 h with (+) or without (−) TrkC antibody. Cell death was monitored by TUNEL labeling of cells platted on slides. TrkC-ICD641N expression was controlled by anti-Cterminal TrkC western blot (lower panel). Representative images are shown on the right panels.

(B) Efficacy of TrkC siRNA was evaluated by western blot on non-expressing TrkC 13.S.24 olfactive neuroblasts. Cells were transfected either with empty vector (control) or with uncleavable TrkC D945N D641N double mutant that does not trigger apoptosis, and with scramble siRNA (siRNA scr.) or TrkC siRNA (siRNA TrkC).

(C) Cell death induction in CLB-Ge2 cell line was quantified after transfection with either scramble siRNA, TrkC siRNA, NT-3 siRNA or a mix of TrkC and NT-3 siRNA, using relative caspase-3 activity assay.

(D) Phospho-Akt and phospho-Erk levels in CLB-Ge2 cells were monitored by western blot after 16 h of treatment with 2 μg/ml α TrkC antibody, 20 nM Ly29402, 100 nM U0126 or 100 ng/ml NT3, in absence of serum.

(E) Detection of TrkC cleavage band (20 kDa, indicated by the arrow) by western blot using an anti-TrkC antibody, on cells treated (or not) with TrkC blocking antibody, with or without the general caspase inhibitor BAF. In A and C, error bars indicate s.e.m.; * indicates a p<0.05 calculated by using a two-sided Mann-Whitney test, compared to control.

FIGS. 21A-21B: NT-3/TrkC interference promotes TrkC pro-apoptotic activity.

(A) CLB-Ge2 cells were transfected with either empty vector (control) or with a plasmid encoding the dominant negative TrkC-IC D641N and treated 24 h with (+) or without (−) TrkC antibody. Cell death was monitored by trypan blue exclusion. Errors bars indicate s.e.m.; * indicates a p<0.01 calculated with a Chi-square test.

(B) IMR32 cells were transiently transfected with a pcDNA control vector, Bax or TrkC expressing construct and cell death was measured using Toxilight assay (left panel) or TUNEL staining (right panel). Representative images are shown (lower panel). Errors bars indicate s.e.m; indicates a p 0.05 calculated by a two-sided Mann-Whitney test.

FIG. 22: Expression profile of NT-3 in breast cancer was examined with quantitative real-time RT-PCR. QRT-PCR was performed by using total RNA extracted from 82 tumor biopsies. They were obtained from patients with tumors localized to the breast (N0), with only axillary node involvement (N+M0), and with distant metastases at diagnosis (M+). Specific human NT-3 primers and primers corresponding to the human HMBS gene (hydroxymethylbilane synthase) were used.

HMBS was used as a reference here because it shows a weak variability at the mRNA level between normal and breast tumoral tissues, as described (de Kok J B, et al. (2005)).

The median of NT-3 expression level has been calculated for each group of samples (N0, N+MO and M+). The table shows the number and the percentage of samples expressing NT-3 at a level corresponding to 2 times the median.

### EXAMPLES

### Material and Methods

A) Cell Cultures, Transfection Procedures, and Receptor Expression.

Transient transfections of Human Embryonic Kidney 293T and olfactory neuroblasts 13.S.24 cells were performed as previously described (4), by using Lipofectamine Plus (Invitrogen, Carlsbad, Calif.) according to the manufacturer's instructions. HEK293T and 13.S.24 cells where cultured in DMEM (Invitrogen) with the addition of 0.1% gentamycin for 13.S.24 cells. The recombinant human NT-3 was purchased from PreproTech (Rocky Hill, N.J.) and added to the culture medium 3 and 24 h after transfection. TrkC expression was monitored by Western blot with an anti-C-terminal antibody purchased from Santa Cruz Biotechnology (sc-139; Santa Cruz, Calif.), 24 h after transfection. Plasma membrane localisation of Trk receptors was performed by FACS analysis. Briefly, 106 cells were transfected and labeled successively with anti-HA antibody (1/100; Sigma, St. Louis, Mo.) and anti-rabbit PE (1/100; Jackson ImmunoResearch Laboratories, West Grove, Pa.). Detection was done using a FACSCalibur (BD Biosciences, San Jose, Calif.).

B) Site-Directed Mutagenesis, Plasmid Construction, and siRNA Design.

The PCMX-Rat HA-TrkA, TrkB, and TrkC plasmids were a gift from S. Meakin (The Robarts Research Institute, London, ON, Canada). A TrkC HindIII/XbaI fragment was cloned into the pCDNA3 vector (Invitrogen). TrkC IC was subcloned into the directional pCDNA3.1 (Invitrogen) by PCR using the following primers: forward 5′-CACC ATG AAC AAG TAC GGT CGA CGG TC-3′ and reverse 5′-CTG GAC ATT CTT GGC TAG TGG-3′. TrkC mutants were obtained by Quickchange (Qiagen, Valencia, Calif.) using the following primers: D641N: 5′-GCG ATG ATC CTT GTG AAT GGA CAG CCA CGC CAG G-3′ and 5′-CCT GGC GTG GCT GTC CAT TCA CAA GGA TCA TCG C-3′; D495N: 5′-ACA CCT TCA TCG CTG AAT GCT GGG CCG GAT AC-3′, and 5′-GTA TCC GGC CCA GCA TTC AGC GAT GAA GGT GT-3′; D679N: 5′-CTT TGT GCA CCG AAA CCT GGC CAC CAGG-3′ and 5′-CCT GGT GGC CAG GTT TCG GTG CAC AAA G-3′. The Ret IC D707N has also been described previously (7). Constructs expressing the different TrkC domains were cloned in pcDNA3.1 directional by PCR using the following primers: 496-642 fragment: forward 5′-CACC ATG GCT GGG CCG GAT ACA GTG G-3′, reverse 5′-TCA ATC CAC AAG GAT CAT CGC ATC-3′; 496-825 fragment: forward 5′-CACC ATG GCT GGG CCG GAT ACA GTG G-3′, reverse 5′-CTA GCC AAG AAT GTC CAG GTA G-3′, 642-825 fragment: forward 5′-GGC CTG GCG TGG CTG TCA ATC CAC AAG GAT CAT C-3′ reverse 5′-CTA GCC AAG AAT GTC CAG GTA G-3′. Rat TrkC in pCDNA3 was used as template. pLenti-humanTrkC was a kind gift from P. Sorensen (University of British Columbia, Vancouver, BC, Canada) and B. Nelkin (The Johns Hopkins University, Baltimore, Md.). Human TrkC was subcloned into directional pCDNA3.1 (Invitrogen) by PCR using the following primers: forward 5′-CG CACC ATG GAT GTC TCT CTT TGC CCAG-3′, reverse 5′-GCG TCT AGA CTA GCC AAG AAT GTC CAG GTA G-3′. PLenti-human TrkC was used as template. Dominant-negative mutants of caspase-2, -8, and -9 expressing constructs and Bcl2 vector were described previously (11, 34). To turn-down TrkC expression in DRG, a mix of three siRNA duplexes (Sigma-Aldrich, St. Louis, Mo.) was used. All of the three duplexes were targeting the 3′UTR region of TrkC to invalidate endogenous TrkC but not the product of the injected plasmid. The primers used were: 1S: UCUCAACUCCUUUCUUCCAUU and 1AS: UGGAAGAAAGGAGUUGAGAUU, 2S: CUCAAGUGCCUGCUACACAUA and 2AS: UGUGUAGCAGGCACUUGAGUA, 3S: GCAUUUAUACUCUGUUGCCUC and 3AS: GGCAACAGAGUAUAAAUGCUC.

C) Cell Death Analysis.

Cell death was analyzed using trypan blue staining procedures, as previously described (6). Either the z-VAD-fmk (TEBU-bio, Le Perray en Yvelines Cedex, France; 20 mM) or the BAF [Boc-Asp(Ome) Fluoromethyl Ketone; Sigma-Aldrich; 20 mM] caspase inhibitors were added to the culture medium just after transfection. The extent of cell death is presented as the percentage of trypan blue-positive cells in the different transfected cell populations. Relative caspase activity was determined by fluorescence measurement of DEVDAFC cleavage, as described in (8). Immunostaining using anti-caspase-3 antibody (Cell Signaling) was described in (4). Staining of caspase activity by flow cytometric analysis was done as follows: 7'105 transfected cells were harvested, washed once in 1 ml PBS, and resuspended in 200 ml staining solution containing FITC-VAD-fmk (5 mM, CaspACE, Promega). After incubation for 1 h at 37° C., cells were washed in 1 ml PBS and resuspended in 300 ml PBS for flow cytometry analysis. Detection of apoptotic cells with monoclonal antibody to single-stranded DNA was done with the anti-ssDNA/APOSTAIN F7-26 anti ssADN antibody (AbCys SA, Paris, France). Three hundred thousand transfected cells were harvested, washed once in 1 ml PBS, and fixed for 1-3 days in methanol at 15-20° C. Fixed cells were then pelleted, resuspended in 250 ml formamide, and kept for 5 min at room temperature. Tubes were then immersed in a water bath preheated to 75° C. for 10 min. Two milliliters of 1% nonfat dry milk in PBS were added to the tubes, which were then vortexed and kept at room temperature for 15 min. After centrifugation, the cell pellet was resuspended in 100 ml of monoclonal antibody (10 mg/ml) and incubated at room temperature for 15 min. The cell pellet was then washed in PBS and resuspended in 100 ml of fluorescein-conjugated anti-mouse IgM and incubated 15 min at room temperature. Cells were then washed in PBS and resuspended in 100 ml PBS for flow cytometry analysis. For flow cytometric analysis, stained cells were counted using a FACSCalibur (BD Biosciences) and CellQuest analysis software with excitation and emission settings of 488 nm and 525-550 nm (filter FL1), respectively.

D) In Vitro Transcription/Translation and Caspase Cleavage Reactions.

Purified caspases were a generous gift from Guy Salvesen (The Burnham Institute, La Jolla, Calif.). In vitro transcription/translation and incubation with caspases-3 or -8 were performed as described previously (6).

E) Cleavage Observation in Cell Lines and in DRG.

To observe the cleavage of TrkC in 13.S.24, MG132 (1 mM, Z-Leu-Leu-Leuala, Sigma-Aldrich) was added to the culture medium 2 h before harvesting the cells. Cells were then potterised in the Wang buffer (20 mM Hepes KOH/10 mMKCl/1.5 mM MgCl2/1 mM Sodium EDTA/1 mM Sodium EGTA/0.1 mM PMSF/1 mM DTT/5 mg/ml pepstatin/10 mg/ml leupeptin/2 mg/ml aprotinin). Proteins were then analyzed by Western blot using the anti-TrkC antibody (sc-139; Santa Cruz Biotechnology). To monitor TrkC cleavage in vivo, E10.5 OF1 mouse embryos were dissected with a sharpened tungsten needle to isolate the spinal cord, the somites and DRG precursors which were then semidissociated in DMEM F-12 (Invitrogen). The samples were incubated overnight under agitation at 37° C. in the presence or absence of either NT-3 (PreproTech; 10 ng/ml), or BAF (Sigma-Aldrich; 20 mM), or the NT-3 blocking antibody AF1404 (R&D, Minneapolis, Minn.; 1/100). The samples were then lysed directly in the Laemmli sample buffer and the proteins separated on 12% SDS/PAGE. The filter was probed with the anti-TrkC antibody. The experiment was repeated three times with similar results.

F) MAPK/PI3K Pathways Activation.

Cells were stimulated or not with different concentrations of NT-3 (Preprotech; 10 min), in presence or in absence of the PI3K inhibitor LY294002 (10 mM, 10 min; Sigma-Aldrich), or MEK inhibitor U0126 (10 mM, 15 min; Promega, Madison, Wis.). Cells were lysed in the following buffer (50 mM Tris pH 7.5/1 mM EDTA/1 mM EGTA/0.5 mM Na3VO4/0.1% Betamercaptoethanol/1% Triton (x100)/50 mM Sodium Fluoride/5 mM Sodium Pyrophosphate/10 mM Betaglycerophosphate/0.1 mM PMSF). Proteins were then analyzed by Western blot using an anti-Erk antibody (Cell Signaling, Technology Danvers, Mass.), an anti-phospho-Erk antibody (Cell Signaling), an anti-Akt antibody (Stressgen, La Jolla, Calif.), or an anti-phospho-Akt antibody (New England Biolabs, Ipswich, Mass.).

G) Sensory Neurons' Dissociation and Culture.

Dorsal root ganglia (DRG) were prepared from NMRI strain mice embryos, treated with 1% trypsin (Worthington, Biochemicals Freehold, N.J.) for 15 min, and dissociated mechanically, essentially as described for trigeminal neurons (7). The normeuronal cells were removed by preplating, and the neurons were grown on polyornithine-laminin-coated dishes with either 10 ng/ml of human NT-3 (PeproTech) or 30 ng/ml of 2.5S mouse NGF (Promega). Three times as many cells were plated for the NT-3 cultures. Five-day-old cultures were used for microinjection. To deprive NT-3, the cultures were washed gently three times with NT-3-free-culture medium. To remove NGF, the cultures were washed once with NGF-free medium and function-blocking anti-NGF antibodies (Roche, Indianapolis, Ind.) were added.

H) Microinjections

The neurons were microinjected essentially as described (4). Briefly, the expression plasmids (50 ng/ml) were injected into the nuclei of the neurons (25-80 neurons per experimental point), and the neurons were grown further without neurotrophic factors. Initial neurons surviving the procedure were counted 4-6 h later. The living healthy neurons were counted 72 h later and expressed as a percentage of initial neurons. The results of three independent experiments were expressed as mean±SEM and analyzed by one-way ANOVA and post hoc Tuckey's honestly significant difference test. Null hypothesis was rejected at P<0.05. For TrkC invalidation in neurons, The 3 TrkC siRNA duplexes were combined as a 6 mM final concentration. Control siRNA (sc-37007; Santa Cruz Biochemicals, Santa Cruz, Calif.) was also injected at 6 mM concentration. TrkC plasmid was 50 ng/ml and GFP plasmid was 5 ng/ml. The injected cultures were grown with NT-3 overnight, then NT-3 was removed and, 72 h later, living and fluorescent neurons were counted.

I) Experimental Cell Procedures (Particularly for Examples 7 to 10)

a) Human NB Tumors Samples and Biological Annotations

Following parents consents, surgical human neuroblastoma tumors material was immediately frozen. Material and annotations were obtained from the Biological Resources Centers of both national referent Institutions for NB treatment, i.e., Centre Leon Berard (Lyon, France) and at Institut Gustave Roussy (Villejuif, France).

b) Cell Line, Transfection Procedure, Reagents

Human neuroblastoma cell lines were from the tumor banks at Centre Leon Berard and at Institut Gustave Roussy. CLB-Ge2, CLB-VolMo and IMR32 cell lines were cultured in RPMI 1640 Glutamax medium (Gibco) containing 10% fetal bovine serum. CLB-Ge2 and IMR32 cells were transfected using lipofectamine 2000 reagent (Invitrogen). Tumor biopsies and bone marrow cells were immediately dissociated and cultured on RPMI 1640 Glutamax medium (Gibco) containing 10% fetal bovine serum. Olfactory neuroblasts 13.5.24 were cultured and transfected as previously described (36). Anti-TrkC blocking antibody (a TrkC) was obtained from R&D Systems (AF1404). Recombinant TrkC/Fc chimera corresponding to extracellular domain of human TrkC (Fc-TrkC-EC) was obtained from R&D Systems (373-TC). BAF [Boc-Asp(Ome) Fluoromethyl Ketone] caspase inhibitor (20 mM) was from Sigma-Aldrich.

c) Plasmid Constructs, siRNA

TrkC dominant negative mutant TrkC-IC D641N and uncleavable TrkC D495N/D641N were described before (36). Scramble siRNA (sc-37007) and NT-3 siRNA (sc-42125) were obtained from Santa Cruz Biotechnology. TrkC siRNA was from Sigma-Aldrich (SASI_Hs01_{—}00192145 and SASI_Hs01_{—}00192145_AS).

d) Cell Death Assays

2×10⁵ cells were grown in serum-poor medium and were treated (or not) with 2 μg/ml of anti-TrkC antibody (R&D Systems AF1404), 2 μg/ml of Fc-TrkC-EC (R&D Systems 373-TC) or transfected with siRNA or TrkC constructs using Lipofectamine 2000 (Invitrogen) for CLB-Ge2 cells or Lipofectamine Plus for IMR32 cells (Invitrogen). Cell death was analyzed 24 h after treatment/transfection either by trypan blue exclusion as described previously (6) or with ToxiLight Bio Assay kit (Lonza). Apoptosis was monitored by measuring caspase-3 activity as described previously (6) using ApoAlert CPP32 kit from Clontech (USA). For detection of DNA fragmentation, CLB-Ge2 cells were grown in Poly-L Lysine coated slides and fixed with 4% paraformaldehyde (PFA) 24 h after treatment/transfection. IMR32 transfected cells were cytospun before PFA fixation. Terminal deoxynucleodityl transferase mediated dUTP-biotin Nick End Labelling (TUNEL) was performed with 300 U/mL TUNEL enzyme (300 U/mL) and 6 μM biotinylated dUTP (Roche Diagnostics), as described previously (39).

e) Quantitative RT-PCR

To assay NT-3 and TrkC expression in neuroblastoma samples, total RNA was extracted from histologically qualified tumor biopsies (>60% immature neuroblasts) using the Nucleospin RNAII kit (Macherey-Nagel) and 200 ng were reverse-transcribed using 1 U Superscript II reverse transcriptase (Invitrogen), 1 U RNAse inhibitor (Roche Applied Science) and 250 ng random hexamer (Roche Applied Science). Total RNA was extracted from human cell lines using the Nucleospin RNAII kit (Macherey-Nagel) and 1 μg was reverse-transcribed using the iScript cDNA Synthesis kit (BioRad). Real-time quantitative RT-PCR was performed on a LightCycler 2.0 apparatus (Roche) using the Light Cycler FastStart DNA Master SYBERGreen I kit (Roche). Quantitative RT-PCR was performed using the primers: TrkC: forward 5′-AGCTCAACAGCCAGAACCTC-3′ and reverse 5′-AACAGCGTTGTCACCCTCTC-3′. NT-3: forward 5′-GAAACGCGATGTAAGGAAGC-3′ and reverse 5′-CCAGCCCACGAGTTTATTGT-3′. The ubiquitously expressed human HPRT genes showing the least variability expression in neuroblastoma was used as an internal control using the following primers: forward 5′-TGACACTGGCAAAACAATGCA-3′ and reverse 5′-GGTCCTTTTCACCAGCAAGCT-3′. For all three couple of primers, polymerase was activated at 95° C. for 10 min followed by 35 cycles at 95° C. for 10 s, 60° C. for 10 s and 72° C. for 5 s.

f) Immunohistochemistry and Immunoblot

8×10⁴ cells were cytospun on coverslips and fixed in 4% PFA. The slides were then incubated at room temperature for one hour with an antibody recognizing human NT-3 (1/300, SC-547). After rinsing in Phosphate Buffer Saline, the slides were incubated with an Alexa-488-Donkey anti-Rabbit antibody (Molecular Probes). Nucleus were visualized with Hoechst staining (Sigma).

Expression of TrkC constructs and endogenous TrkC cleavage were monitored by western blot with anti-Trk antibody (sc-11; Santa Cruz Biotechnology) and an anti-actin (13E5; Cell Signaling) was used as loading control as previously described (36).

Phospho-Akt and phospho-Erk levels of CLB-Ge2 cells were measured by western blot with anti-phospho-Akt (4058, Cell Signaling) and phospho-Erk1 &Erk2 (E7028, Sigma) after 16 h of culture on serum free medium with 2 μg/ml anti-TrkC antibody (R&D Systems AF1404), 20 nM Ly29402 (Sigma), 100 nM U0126 (Sigma) or 100 ng/ml NT3 (Abcys).

g) Chicken Model for NB Progression and Dissemination

10⁷ neuroblastoma cells suspended in 40 μL complete medium were seeded on 10-day-old (day 10) chick chorioallantoic membrane (CAM). 2 μg of anti-TrkC antibody or an isotypic unrelated antibody (Santa Cruz Biotechnology sc-1290) were injected in the tumor on day 11 and day 14. For siRNA treatment, 3 μg of scramble, TrkC or NT-3 siRNA were injected in a chorioallantoic vessel on days 11 and 14. On day 17, tumors were resected and area measured with AxioVision Release 4.6 software. To monitor apoptosis on primary tumors, they were fixed on 4% PFA, then cryoprotected by overnight treatment with 30% sucrose and embedded in Cryomount (Histolab). TUNEL staining was performed on tumor cryostat sections (Roche Diagnostics) and nucleus were stained with Hoechst. To assess metastasis, lungs were harvested from the tumor-bearing embryos and genomic DNA was extracted with NucleoSpin Tissue kit (Macherey Nagel). Metastasis was quantified by RT-Q-PCR detection of the human Alu sequence using the following primers: forward 5′-ACGCCTGTAATCCCAGCACTT-3′ and reverse 5′-TCGCCCAGGCTGGAGTGCA-3′. Chick glyceraldehyde-3-phosphate dehydrogenase (GAPDH) specific primers were used as controls: forward 5′-GAGGAAAGGTCGCCTGGTGGATCG-3′; reverse 5′-GGTGAGGACAAGCAGTGAGGAACG-3′. For both couple of primers, metastasis invasion was assessed by polymerase activation at 95° C. for 2 min followed by 30 cycles at 95° C. for 30 s, 63° C. for 30 s and 72° C. for 30 s. Genomic DNA extracted from lungs of non inoculated chick embryos were used to determine the threshold.

### Example 1

### TrkC is a Dependence Receptor

We first transiently expressed full-length rat TrkC in HEK293T cells (in which HEK stands for “human embryonic kidney”) or in immortalized olfactory neuroblast 13.5.24 cells. TrkC expression was detected only when these cells were transfected with a TrkC-encoding construct [supporting information (SI) FIG. 5 and FIGS. 1A and 1F]. As shown in FIG. 1A, cell death induction was associated with the expression of TrkC. TrkC-induced cell death was defined as apoptosis because TrkC expression induced (i) an increased caspase activity [determined by the measurement of DEVD-AFC cleavage in cell lysate (FIG. 1B), by the quantification of cells stained with anti-active caspase-3 antibody (FIG. 1C), or by measuring the cleavage of a FITC-VAD-fmk caspase substrate in living cells (FIG. 1D)] and (ii) an increased DNA condensation [determined by the percentage of cells stained with an anti-single stranded DNA antibody (SI FIG. 5B)]. This apoptosis is caspase-dependent because addition of the general caspase inhibitors zVAD-fmk or boc-aspartyl(OMe)-fluoromethylketone (BAF) fully inhibit TrkC-induced apoptosis (FIG. 1B and data not shown). Interestingly, such a death-promoting effect is not observed when TrkA or TrkB is expressed instead of TrkC (FIG. 1D), even though TrkA, TrkB, and TrkC are present at the cell membrane at a similar level (SI FIG. 5A and data not shown). To exclude the possibility that apoptosis induction could be caused by abnormal autoactivation of TrkC, a kinase-dead mutant, TrkC D679N, was expressed instead of TrkC wild type. This mutant, which fails to induce Erk or Akt phosphorylation in response to NT-3 (see FIG. 4C), displays a similar proapoptotic activity to TrkC wild type (FIG. 1D). Thus, TrkC expression drives apoptotic cell death that is not caused by TrkC kinase activity.

We then assessed whether the presence of NT-3 affected TrkC-proapoptotic activity. TrkC-mediated cell death [measured by the trypan blue exclusion assay (FIG. 1E), by caspase activity (FIG. 1F), or by DNA condensation (SI FIG. 5C)] was inhibited, in a dose-dependent manner, by NT-3 used within the range of NT-3 concentration that triggered the classic positive signaling downstream of TrkC [i.e., measured by Akt or Erk phosphorylation (FIG. 1E)]. Hence, NT-3 blocks TrkC-mediated apoptosis. Moreover, the dependence effect is not restricted to rat TrkC; human TrkC also triggers cell death unless NT-3 is present (FIG. 1G). Taken together, these data show that TrkC acts as a dependence receptor.

### Example 2

### TrkC Intracellular Domain is Cleaved by Caspase

To elucidate the molecular mechanisms of TrkC-induced cell death, we further analyzed the involvement of caspases. The dependence receptors DCC, UNC5H, Patched, and RET were shown to require preliminary caspase cleavage to induce cell death (4, 6-8). We therefore analyzed whether the intracellular domain of TrkC can be cleaved by caspases. The intracellular region of TrkC encompasses the last 372 C-terminal amino acids. This domain was translated in vitro, and the product was incubated with purified active caspase-3 or caspase-8. FIG. 2A shows that the intracellular domain of TrkC is cleaved in vitro by caspase-3 but not by caspase-8. In the same experimental conditions, TrkA and TrkB intracellular domains failed to be significantly cleaved by caspase-3 (FIG. 2A). Hence, TrkC is cleaved in vitro by caspases and particularly by caspase-3-like caspases. Incubation with active caspase-3 leads to the detection of cleavage products that migrate at apparent relative molecular masses of 19, 15, and 6 kDa, suggesting the presence of at least two sites of cleavage. The caspase cleavage sites were mapped by constructing mutants based on preferred P4 and P1′ positions (9) and the apparent relative sizes of the caspase cleavage fragments. Whereas mutation of various aspartic acid (Asp) residues within the intracellular domain of TrkC had no effect on caspase-3 cleavage, the mutation of Asp-641 to Asn completely suppressed the appearance of the 19- and 15-kDa fragments (FIG. 2B). The second caspase site was subsequently located at Asp-495, because the double mutant D641N and D495N was completely resistant to caspase-3 cleavage (FIG. 2B). Thus, TrkC is cleaved by caspases at two sites located at Asp-495 and Asp-641. Interestingly, these aspartic residues appear to be conserved in chick, rat, mouse, and human TrkC, but they were not found at the corresponding positions in TrkA or TrkB. An immunoblot performed on 13.5.24 cells expressing TrkC, by using an antibody raised against a TrkC C-terminal epitope, revealed two bands (around, respectively, 35 and 20 kDa) that failed to be detected when cells were treated with the general caspase inhibitors zVAD-fmk (FIG. 2C) and BAF (SI FIG. 6A). The same two bands were observed when human TrkC was expressed in 13.S.24 cells (SI FIG. 6C). Moreover, the mutation of D495N inhibits the appearance of the 35-kDa fragment (mutation of D641N is associated with the absence of the 20-kDa band), whereas the double mutant expressed in 13.S.24 cells fails to show either of these two fragments (FIG. 2C). Thus, these two bands represent two TrkC fragments resulting from the endogenous caspase cleavage of TrkC at Asp-495 and Asp-641. Interestingly, this caspase cleavage at the two sites is not affected by overexpression of the Trk coreceptor p75 ntr (SI FIG. 6D). Yet, the nature of the TrkC-cleaving caspase remains to be shown. Indeed, if in vitro caspase-3 cleaves TrkC, it is probably not only caspase-3 that cleaves TrkC in cells; both caspase-3 inhibitor DEVD-fmk and the use of a dominant-negative mutant for caspase-3 fail to block caspase-dependent cleavage of TrkC in 13.S.24 cells (SI FIGS. 6A and 6B). To monitor whether the TrkC cleavage by caspases naturally occurs, embryonic mouse dorsal root ganglion (DRG) were semidissociated and maintained overnight in the presence of 10 ng/ml NT-3 or in the presence of a TrkC-blocking antibody together or not with the caspase inhibitor BAF. Whereas a 20-kDa band was detected in normal culture condition, this TrkC fragment disappeared with NT-3 or BAF while it was enhanced by the blocking antibody presence (FIG. 2D). Together, these data support that TrkC is cleaved by caspases in cell-free conditions, in transfected cells, and in DRG.

### Example 3

### Caspase Cleavage of TrkC Releases a TrkC-Proapoptotic Domain

To evaluate the functional importance of the cleavage of the TrkC protein by caspases, we expressed the full-length TrkC D641N mutant, the TrkC D495N mutant, or the TrkC D641N/D495N double mutant in 13.S.24 or HEK293T cells, and cell death was assessed by trypan blue exclusion assay, and by measuring caspase activity or DNA condensation (FIGS. 3A-3D). Remarkably, although the mutations of one single caspase site and both caspase sites failed to affect expression levels and plasma membrane localization of TrkC (FIG. 3A and data not shown), they were sufficient to fully inhibit TrkC-proapoptotic activity (FIGS. 3B-3D). Taken together, these results indicate that the caspase cleavage of TrkC is a prerequisite for TrkC-proapoptotic activity. We next investigated whether this cleavage allows the release or the exposure of a proapoptotic domain (i.e., the dependence domain). The deletion of the region located after Asp-495 was sufficient to abrogate TrkC-proapoptotic activity (data not shown). We then expressed the complete intracellular domain, the region located after the second caspase cleavage site Asp-641, or the fragment encompassed between the two caspase cleavage sites in 13.S.24 cells. As shown in FIGS. 3F and 3G, expression of the fragment located between Asp-495 and Asp-641 (FIG. 3E) was sufficient to trigger apoptosis, whereas the 642-825 fragment failed to display any proapoptotic activity. Intriguingly, TrkC intracellular domain expression failed to induce apoptosis, whereas the full-length TrkC was proapoptotic, suggesting that the caspase cleavage and the subsequent cell death induction requires transmembrane TrkC (FIGS. 3F and 3G). Moreover, together with the observation that the mutation of one single caspase cleavage site is sufficient to abrogate TrkC-proapoptotic activity, the fact that the fragment resulting from the two caspase cleavages (i.e., TrkC 496-641) kills cells, whereas the fragment resulting from the single caspase cleavage at Asp-495 (for example, TrkC 496-825) does not, supports the argument that both caspase cleavages are required for TrkC-induced apoptosis (FIG. 3F).

To monitor whether this fragment was proapoptotic in a more biological setting, we analyzed whether expression of this dependence domain of TrkC (TrkC 496-641) was proapoptotic in TrkC expressing primary neurons. We analyzed embryonic mouse DRG neurons maintained in culture for 5 days with NT-3 and then as a control deprived of NT-3 (also see FIG. 4). As shown in FIG. 3H, expression of this domain via microinjection was apoptotic in NT-3-maintained DRG neurons, hence surpassing the survival signaling provided by NT-3. Together with the fact that NT-3 inhibits caspase-dependent TrkC cleavage in DRG (FIG. 2D), this observation supports the view of unbound TrkC being cleaved by caspases, resulting in the release of a TrkC-proapoptotic fragment. How the released fragment induces apoptosis remains to be shown. However, it is interesting to note that death induction by this fragment resembles death induction by DCC, another dependence receptor (6). Indeed, TrkC dependence domain-induced 13.S.24 cell death appears independent of the death-receptor pathway because expression of a dominant-negative mutant of caspase-8 that is known to block TNF- or Fas-induced cell death failed to inhibit TrkC-496-641-induced cell death (FIG. 3I). Similarly, TrkC dependence domain-induced cell death was not inhibited by the dominant-negative mutant of another initiator caspase, caspase-2 (FIG. 3I). On the contrary, caspase-9 dominant-negative mutant fully inhibited cell death induced by the TrkC dependence domain (FIG. 3I). The requirement of caspase-9 was rather suggestive of the involvement of the mitochondrial apoptotic pathway. Yet, we failed to observe inhibition of 13.S.24 cell death when Bcl2 was overexpressed (FIG. 3I), hence suggesting that this released domain does not kill through the mitochondria-dependent pathway. This finding is in agreement with the observation that Bcl-XL overexpression failed to block the death of cultured DRG neurons associated with NT-3 withdrawal (L.-Y.Y. and U.A., unpublished data). However, this observation is not supported by the phenotype of NT-3/Bax double knockout mice that show survival of proprioceptive neurons, suggesting a more complex regulation of neuronal death in vivo (10). Even though a more detailed study on the mechanisms used by the TrkC dependence domain to kill cells in vivo and in vitro remains to be done, it is intriguing to relate TrkC-induced cell death with DCC-induced cell death that requires (i) DCC cleavage by caspase, (ii) the release/exposure of a proapoptotic dependence domain, and (iii) interaction of this domain with caspase-9 and activation of caspase-9 (11). Whether the dependence domain of TrkC recruits caspase-9 and activates apoptosis through such a caspase-activating complex remains, however, to be shown.

### Example 5

### The Dependence Receptor Activity of TrkC is a Prerequisite for Sensory Neuron Death

We then investigated whether the TrkC-proapoptotic activity described here has any implication in the death of primary neurons after withdrawal of NT-3. As also observed with the dependence receptor Patched (Ptc), we first noticed that the expression of a mutant form of TrkC [i.e., the intracellular domain of TrkC bearing a mutation on the caspase site D641 (TrkC IC D641N)] completely inhibits cell death induced by full-length TrkC (FIGS. 4A and 4B). This dominant-negative effect was specific, because the expression of TrkC IC D641N had no effect on Ptc- or Bax-induced apoptosis (data not shown). Thus, TrkC IC D641N acts as a specific dominant-negative mutant for TrkC-proapoptotic activity. The D641N mutation could theoretically lead to ectopic activation of the TrkC kinase domain when the intracellular region is separated from the whole receptor, and the resulting enhanced survival signaling could prevent apoptosis. To exclude this possibility, we analyzed Erk and Akt phosphorylation in response to NT-3 treatment in TrkC-transfected 13.S.24 cells. As shown in FIG. 4C, the presence of the TrkC IC D641N dominant-negative mutant does not induce activation of Erk/Akt in the absence of NT-3, nor does it interfere with NT-3-dependent TrkC-mediated Erk/Akt activation. Thus, the TrkC IC D641N does not prevent the death via increased survival signaling but, instead, via interfering with death signaling activated by deliganded TrkC.

To check the antiapoptotic effect of TrkC IC D641N on endogenous TrkC, we dissociated DRG from embryonic mice and cultured sensory neurons in the presence of NT-3 for 5 days. Control neurons were maintained with NGF that activates TrkA. Withdrawal of either NGF or NT-3 leads to death of ≈60-70% of the neurons, upon being counted 72 h later. We microinjected the NT-3- or NGF-maintained neurons with either TrkC IC D641N or the mock vector and removed NT-3 or NGF. As shown in FIG. 4D, the dominant-negative mutant dramatically enhanced survival of the NT-3-deprived neurons, although it did not affect the death of NGF-deprived neurons. Interestingly, microinjection of a construct encoding the intracellular domain of TrkC without the D641N mutation had no effect on survival of the NT-3-deprived neurons (FIG. 4E). Thus, the antiapoptotic effect of TrkC IC D641N on NT-3-deprived neurons is not due to overexpression of the ectopic TrkC intracellular domain, which may have forced TrkC kinase catalytic activity. To specifically exclude the role of the tyrosine kinase domain, we microinjected the TrkC IC D641N bearing the additional kinase-inactivating mutation D679N (this mutation abrogates TrkC ability to activate Erk or Akt in response to NT-3; see FIG. 4C). As shown in FIG. 4E, the kinase-inactivating mutation did not abolish the death-suppressing activity of TrkC IC D641N in NT-3-deprived neurons, showing that the tyrosine kinase catalytic activity is not involved here. Moreover, the antiapoptotic effect of TrkC IC D641N on NT-3-deprived neurons is receptor-specific, because the microinjection of a dominant-negative mutant of another dependence receptor, Ret (Ret IC D707N), into NT-3-deprived (and also NGF-deprived) neurons failed to inhibit death (FIG. 4E). To further study whether cell death observed upon NT-3 loss is related to the endogenous proapoptotic activity of unbound TrkC, we performed a replacement study in which endogenous TrkC was inhibited via microinjection of siRNA while ectopic TrkC wild type or TrkC mutated in the two caspase sites TrkC D495N/D641N was expressed. As control experiments, microinjection of control siRNA failed to have significant effect on primary neurons death upon NT-3 loss (data not shown). Moreover, as shown in FIG. 4F, similarly to the control situation, replacement of endogenous TrkC by ectopic TrkC is associated with primary neuron death in response to NT-3 loss. On the other hand, the replacement of endogenous TrkC by the TrkC caspase-dead mutant inhibited cell death induction upon NT-3 withdrawal (FIG. 4F). Moreover, this effect is not due to a possible interference of the caspase site's mutation with TrkC positive signaling, because TrkC wild type and TrkC D495N/D641N show a similar pattern of Akt/Erk phosphorylation in the absence or presence of NT-3 (FIG. 4G). Taken together, these data demonstrate that the cell death observed upon NT-3 loss is not only due to the loss of survival signals but also to an active cell death stimulus triggered by unbound TrkC.

Discussion

Many neurons die physiologically in vivo at different stages of development, a process in which neurotrophins and their receptors play a key role. In developing sensory ganglia, NT-3-dependent neurons are overproduced. Excess neurons are removed through a deficiency in NT-3 during periods of programmed death. Along this line, overexpression of NT-3 in mouse increases the number of neurons in DRG (12-14). The classic view proposes that the death of these neurons is due to loss of the survival signals (i.e., MAPK and/or PI3K pathways) resulting from the loss of kinase activation of neurotrophins receptors. Yet, from our study, it appears tempting to speculate that excess TrkC-expressing NT-3-sensitive neurons die not only because of the loss of these survival signals but also via the unbound TrkC-triggered proapoptotic pathway described here. One interesting hint that fits with this hypothesis is provided by the data obtained from the different knockout mice for neurotrophins and their respective receptors. Indeed, inactivation of TrkA or NGF in mouse results in the same amount of sensory neurons loss at birth (i.e., nociceptive neurons) (15). Similarly, inactivation of either TrkB or BDNF results in an equivalent loss of mechanoceptive neurons (16, 17). On the other hand, neonates invalidated for TrkC present a loss of 30% DRG neurons, whereas NT-3−/− neonates have lost 70% of them (18, 19). The search for an explanation that may fit with the classic view of neurotrophins acting only positively via kinase-dependent signaling has raised a controversy and two hypotheses are currently proposed. Farinas et al. (20, 21) suggested that the increased loss of DRG neurons in NT-3−/− mice is explained by the ability of NT-3 to signal through TrkA and TrkB (20-22). Alternatively, Ernfors et al. (17) have proposed that the effect is due to the death of neuronal precursors that in their vast majority express TrkC early during gangliogenesis before the different subpopulations are established (24). Yet, an alternative and attractive explanation for this discrepancy between TrkC and NT-3 inactivation could be related to the dependence-receptor facet of TrkC. Indeed, as a common feature of dependence receptors, it has been postulated that inactivation of the ligand of a dependence receptor should be associated with a more profound phenotype than inactivation of the receptor. This discrepency has been further demonstrated for the dependence receptor neogenin (25). In the case of TrkC, neuronal death observed in TrkC mutant mice could then be the result of the loss of the positive/kinase signaling of TrkC, whereas neuronal death observed in NT-3 mutant would be the result of both the loss of the positive pathway and the constitutive proapoptotic activity of TrkC. Such a view would be proven, per se, if the double NT-3/TrkC mutant mice show a less-severe phenotype than NT-3 mutant mice. This possibility needs to be further investigated.

Even though it appears clear that more in vivo data are required to apprehend the relative importance of the loss of survival signals and of the active proapoptotic signal initiated by unbound TrkC, this work brings a twist in the neurotrophic theory that assumes that the loss of neurotrophic factors equals the loss of survival signals, leading to “death by default”. Here, we propose that the mere loss of survival signals is not sufficient to explain physiological neuronal death. An active proapoptotic activity is also necessary to create the “intrinsic apoptotic status” when neurotrophic support is inadequate. In some cases, this active proapoptotic signal is provided by dependence receptor-independent mechanisms, such as the stimulation of p75 ntr by unprocessed pro-NGF (26, 27), or the engagement of the Fas receptor by the Fas ligand (28). However, in several cases, as shown here in NT-3-dependent sensory neurons, such proapoptotic activity could be mediated by the unbound dependence receptor TrkC.

However, it can be argued that TrkC, which requires a caspase cleavage to be proapoptotic, is not sufficient to trigger apoptosis by itself but rather acts as an amplifier downstream of a primary apoptotic stimulus. Indeed, how can a receptor initiate apoptosis while it requires, to produce a proapoptotic molecule, a cleavage by caspases that are believed to be the effectors of apoptosis. One possibility is that the process may be initiated by a noncaspase protease, then propagated via caspase cleavage. Only a few cleavage events by a noncaspase protease would then be sufficient to initiate the cell death pathway by locally activating enough caspase to generate a caspase-amplification loop via these receptors. Alternatively, the now-old dogma suggesting that caspases are completely inactive in nonapoptotic cells and are only activated massively upon proapoptotic stimuli might be wrong. Recent findings have shown that caspase zymogens display some protease activity (29), whereas cells express endogenous caspase inhibitors, such as IAP proteins, that prevent the propagation of active caspases. Similarly, local caspase activation without cell death induction is now being documented (30, 31). Cell-death induction could therefore result from caspase amplification rather than from caspase initiation, and this would support the importance of the cellular control of caspase activation/inhibition in cell-fate determination: cell death induction would be the result of a move from low/local caspase activation (i.e., that may have a “positive” input on the cell like cell differentiation) (30) to high/distributed caspase activation. The balance between low/local and high/distributed caspase activation would therefore likely be modulated by endogenous caspase inhibitors such as IAPs and by endogenous caspase amplifiers such as the dependence receptor TrkC.

Interestingly, TrkA- and TrkB-forced expression failed to induce apoptotic death of HEK293T or 13.S.24 cells. Moreover, TrkA and TrkB are not cleaved by caspases in vitro. Thus, whereas TrkC is a prototype dependence receptor, TrkA and TrkB are probably not, suggesting that even closely related receptors like TrkA, TrkB, and TrkC can acquire a completely different activity regarding cell survival/cell death. Beside mono-sided receptors like TrkA and TrkB, which induce only survival when liganded, two-sided receptors like TrkC control both survival and death. It is tempting to speculate that both sides of the TrkC/NT-3 pair play important roles during development of the nervous system. Whereas the positive signaling pathways activated by TrkC upon NT-3 binding are important for cell differentiation, proliferation, or survival, the negative signaling pathway initiated by TrkC in the absence of NT-3 may be part of the normal apoptotic removal of cells during embryogenesis and adult tissue homeostasis.

TrkC is also involved in tumor formation and especially in medulloblastoma. In particular, elevated expression of TrkC by childhood medulloblastomas is associated with favorable clinical outcome, and it has been proposed that this effect may be related to the ability of TrkC to trigger apoptosis. Indeed, overexpression of TrkC inhibits the growth of intracerebral xenografts of a medulloblastoma cell line in nude mice, and TrkC expression by individual tumor cells is highly correlated with apoptosis within primary medulloblastoma biopsy specimens (32, 33). Even though, to date, this implication has been seen under a classic scheme of a receptor activated by its ligand NT-3, it may be worth considering the importance of the dependence receptor side of TrkC in medulloblastoma development. Moreover, the data presented here with the TrkC tyrosine kinase receptor, which will possibly hold for some other tyrosine kinase receptors, also raises questions about the common anticancer strategy, which is based on inhibiting survival pathways by interfering with the kinase activity of receptors. According to our data, inhibiting the kinase activity may not be sufficient to efficiently trigger death of tumor cells. Thus, a cotreatment based on both kinase inhibition and stimulation of the proapoptotic activity of these tyrosine kinase dependence receptors could appear as a more attractive and efficient therapeutic strategy that may bypass some of the currently observed tumor resistance.

### Example 6

### Inhibition of NT-3/TrkC Interaction Triggers Apoptosis of Neuroblastoma Cells and is Associated with Inhibition of Primary Tumor Growth and Metastasis

In Examples 1-5, we have shown that the neurotrophin receptor TrkC is a dependence receptor and, as such, it induces apoptotic death in the absence of its ligand, neurotrophin-3 (NT-3). This activity relies on the caspase-mediated cleavage of the intracellular domain of TrkC, which allows the release of a proapoptotic fragment. Dependence receptors have been proposed to act as tumour suppressors by inducing apoptosis of tumour cells that grow or migrate beyond the regions of ligand availability. A selective advantage for a tumor cell is then either to lose receptor expression and in this line TrkC expression has been correlated with good prognosis of neuroblastoma (NB) or to gain overexpression of the ligand. We have investigated here whether in some NB, NT-3 could be upregulated and whether this autocrine production of NT-3 could be used as a tool to trigger tumor inhibition.

1) Methods

We measured TrkC and NT-3 expression in 26 human neuroblastoma biopsies by Q-RT-PCR and we have observed that the more aggressive and metastatic NBs (stage 4) show the highest NT3/TrkC ratio (FIG. 10). We selected two cell lines with high NT-3 levels (CLB-Vol and CLB-Ge1) and one NT-3 negative cell line as a control (IMR32 cells) based on Q-RT-PCR (left panel FIG. 11) and this was verified at the protein level by NT-3 immunocytochemistry (right panel FIG. 11). We incubated these cells in presence of an NT-3 antibody antagonizing TrkC-NT3 bound (AF1404) and we measured cell death induction by trypan bleu exclusion and caspase 3 activation. We also set up a model of neuroblastoma development in chicken embryos to evaluate primary tumor growth and metastasis. In this model, we treated tumors with TrkC-blocking antibody. We thus analyzed TrkC proapoptotic activity as a mechanism of control of neuroblastoma, when it can no longer interact with its ligand.

2) Results

TrkC induces apoptosis in NT-3 expressing neuroblastoma cell lines, when incubated in presence of TrkC-blocking antibody while it has no effect on NT-3 negative cells. Similar results where obtained on NB directly cultured from a Stage 4 NB bearing patient. Preliminary results also showed reduced primary tumor development, and inhibition of metastasis in vivo, upon antibody treatment, while no effects where observed with control antibody treatment.

### Example 7

### NT-3 is Expressed in a Large Fraction of Aggressive Neuroblastomas

We focused on stage 4 NB with a specific interest in comparing NT-3 and its receptor TrkC expression levels. We first analyzed the expression of NT-3 and TrkC by Q-RT-PCR in a panel of 106 stage 4 NB tumors. NT-3 is up-regulated in a significant fraction of stage 4 NB (FIG. 16A and FIG. 17). 38% of tumors showed at least a two-fold increase in NT-3 expression compared to the median value, more than 20% displayed a five-fold increase (FIG. 16A, upper graph). Tumors with high NT-3 level showed a high NT-3/TrkC ratio, supporting the view of a gain of NT-3 in tumors (FIG. 16A, lower graph). When comparing NT-3 levels to the prognosis and the different sub-categories of stage 4 NB-stage 4S, stage 4 diagnosed before 1 year of age or later—, no significant differences were observed, suggesting that NT-3 up-regulation is a selective gain that occurs independently of tumor aggressiveness and dissemination in a large fraction of stage 4 NB. Similar results were obtained on stage 1, 2 or 3 NB (FIG. 17). Expression of NT-3 was not only detected at the mRNA level but also at the protein level by immunohistochemistry (FIG. 16B).

NT-3 overexpression is seen in 38% of stage 4 NB but also in a fraction of NB cell lines mainly derived from stage 4 NB tumor material (FIG. 16C). Three human NB cell lines—i.e., CLB-Ge2, CLB-VolMo and IMR32—were studied further. All three cell lines express TrkC (not shown) but CLB-Ge2 and CLB-VolMo express high levels of NT-3 whereas NT-3 was barely detected in IMR32 cells, both at the messengers level (FIG. 16C) and at the protein level (FIG. 16D). Interestingly, NT-3 immunostaining performed on CLB-Ge2 cells in the absence of cell permeabilization showed a clear membranous staining, indicating that the high NT-3 content observed in aggressive NB is associated with an autocrine expression of NT-3 in NB cells.

### Example 8

### NT-3 Expression in Neuroblastoma is a Survival Selective Advantage

To investigate whether the NT-3 autocrine expression observed in CLB-Ge2 and CLB-VolMo cells provides a selective advantage for tumor cell survival, as would be expected from the dependence receptor paradigm, cell death was analyzed in response to the disruption of this autocrine loop. As a first approach, NT-3 was down-regulated by RNA interference. NT-3 siRNA transfection of CLB-Ge2 cells was associated with a significant reduction of NT-3 protein, as observed by immunohistochemistry (FIG. 18A). While scrambled siRNA failed to affect IMR32 and CLB-Ge2 cell survival, as measured by caspase activity (FIG. 18B) or toxilight (FIG. 18C) assays, NT-3 siRNA transfection was associated with CLB-Ge2 cell death (FIGS. 18B and 18C). In contrast, IMR32 cell survival was unaffected after NT-3 siRNA treatment (FIGS. 18B and 18C).

As a second approach, we used a blocking TrkC antibody described before (Tauszig-Delamasure et al., 2007) to prevent NT-3 from binding to endogenous TrkC. As shown in FIGS. 18D and 18E, the addition of anti-TrkC triggered CLB-Ge2 and CLB-VolMo apoptotic cell death, as measured by caspase-3 activity assay (FIG. 18D) and TUNEL staining (FIG. 18E). This effect was specific for NT-3/TrkC inhibition, since the anti-TrkC antibody had no effect on IMR32 cells. Similar CLB-Ge2 cell death induction was observed when, instead of using a blocking TrkC antibody, a recombinant ectodomain of TrkC was used to trap NT-3 (FIG. 19A). To determine whether the NB cell death associated with inhibition of TrkC/NT-3 interaction can be extended to fresh tumors, a surgical biopsy from a tumor and the corresponding invaded bone marrow were semi-dissociated and further incubated with the anti-TrkC antibody. This primary tumor and the disseminated neoplasia express both NT-3 and TrkC (FIG. 19B) and an increased cell death measured by caspase activation was detected in response to the anti-TrkC antibody (FIG. 18F).

### Example 9

### Interference with the NT-3/TrkC Interaction Triggers TrkC-Mediated Cell Death

There are two different ways for interpreting cell death associated with the interference with the NT-3/TrkC interaction. According to the classic neurotrophic view, the observed cell death could be a death by “default” that results from the loss of survival signals triggered by NT-3/TrkC interaction—i.e., MAPK or PI3K pathways activated through TrkC's kinase activity—. The dependence receptor notion offers a different perspective, more compatible with the fact that TrkC expression is usually a good prognosis factor. In this scenario, blocking the interaction between NT-3 and TrkC leads to unbound TrkC actively triggering apoptosis. As a first approach to discard between these two possibilities, NB cell death was induced via anti-TrkC antibody treatment after CLB-Ge2 cells transfection with a dominant negative mutant for TrkC. This dominant negative mutant, TrkC-IC D641N, has been shown to specifically inhibit the pro-apoptotic signaling of unbound TrkC, without affecting its kinase-dependent signaling (36). Expression of the dominant negative mutant fully blocks anti-TrkC-mediated CLB-Ge2 cell death (FIG. 20A and FIG. 21A). To further support this observation, we assessed the extend of CLB-Ge2 cell death associated with NT-3 siRNA in settings of down-regulation of TrkC by siRNA. As shown in FIGS. 21B and 21C, while down-regulation of TrkC in CLB-Ge2 cells is not associated with cell death as would be expected by the classic loss of survival signaling pathways, this down-regulation fully blocks cell death induced by NT-3 siRNA, thus, demonstrating that NT-3 up-regulation observed in CLB-Ge2 cells inhibits the pro-apoptotic signaling triggered by TrkC itself. Along this line, addition of the blocking TrkC antibody to CLB-Ge2 fails to be associated with a decrease in the classic survival pathways as exemplified here by measurement of ERK or Akt phosphorylation (FIG. 21D). Moreover, TrkC caspase cleavage is enhanced by the TrkC blocking antibody (FIG. 21E). Indeed, as previously described, TrkC, and dependence receptors in general, are cleaved by caspase and this cleavage is a pre-requisite for their pro-apoptotic activity (36, 37). As shown in FIG. 21E, while a basal level of TrkC cleavage is detected in control conditions, addition of the blocking antibody is associated with increased TrkC cleavage, a cleavage blocked by addition of the general and potent caspase inhibitor, BAF. Together, these data demonstrate that NT-3 up-regulation observed in NB cells inhibits the pro-apoptotic signaling triggered by the dependence receptor TrkC.

Intriguingly, while CLB-Ge2 cells have selected NT-3 up-regulation to inhibit TrkC-induced apoptosis, IMR32 cells do not express NT-3 and yet are derived from aggressive NB. We thus investigated whether, as expected from the dependence receptor hypothesis, IMR32 cells have selected resistance to TrkC-induced apoptosis through a different mean. As shown in FIG. 22B, while transient transfection of Bax, the general cell death inducer, is associated with IMR32 apoptosis, transient transfection of TrkC failed to trigger IMR32 cell death. Thus, while a large fraction of NB have up-regulated NT-3 to prevent TrkC-induced apoptosis, other NB cells have counter-selected this cell death pathway by other means including inactivation of TrkC death signaling.

### Example 10

### Interference with the NT-3/TrkC Interaction Inhibits NB Progression and Dissemination

We next assessed whether in vivo interference with NT-3/TrkC could be used to limit/inhibit NB progression and dissemination in NB cells with high NT-3 expression. A chicken model, in which grafts of NB cells in the chorioallantoic membrane (CAM) of 10-day-old chick embryos recapitulates both tumor growth at a primary site—within the CAM—as well as tumor invasion and dissemination at a secondary site—metastasis to the lung—has been developed (38, FIG. 15A). In a first approach, CLB-Ge2 or IMR32 cells were loaded in 10-day-old CAM and embryos were treated on day 11 and day 14 with anti-TrkC or an unrelated antibody. 17-day-old chicks were then analyzed for primary tumor growth and metastasis to the lung. As shown in FIGS. 15B and 15D, treatment with the anti-TrkC antibody significantly reduced primary tumor size specifically in CLB-Ge2-grafted CAM, while an unrelated isotopic antibody had no effect. This size reduction was associated with increased tumor cell apoptosis, as shown by an increased TUNEL staining in the tumors treated with anti-TrkC (FIG. 15C). More importantly, anti-TrkC also reduced lung metastasis formation in CLB-Ge2 grafted embryos (but not in IMR32 grafted embryos), as shown in FIG. 15E. To analyze whether the anti-tumor effect observed was specifically due to inhibition of NT-3 interaction with TrkC, primary tumor growth of CLB-Ge2-grafted CAM was analyzed upon repeated intravenous injection of NT-3 siRNA. As shown in FIG. 15F, NT-3 siRNA injection led to a significant decrease in primary tumor size compared to scramble siRNA. Of interest, when TrkC siRNA was used instead of NT-3 siRNA, no significant change over the scramble siRNA was observed. This result strengthens the view that tumor regression effects observed after either prevention of NT-3 binding or NT-3 inhibition is due to an active death signaling mediated by unbound TrkC, as opposed to a consequence of the loss of classic intracellular prosurvival signaling.

### Example 11

### NT-3 is Overexpressed Metastatic Breast Tumors (See FIG. 22)

FIG. 22 shows that NT-3 is overexpressed in 58% of metastatic breast tumors.

The dependence receptor TrkC acts as a conditional tumor suppressor that regulates survival and invasive capacity of NB cells. This ability to specifically induce apoptosis depends on ligand availability. We observed an elevated NT-3:TrkC ratio expression on poor prognosis NB that could confer a selective advantage to cancer cells since they escape to apoptosis induced by TrkC. NB is one of the most common pediatric solid tumors, even though, molecular basis are barely understood and due to its heterogeneity the treatment remains mainly by surgery and chemotherapy. Targeting NT-3 or TrkC by blocking NT-3 binding could lead to an alternative/supplementary therapy for poor prognosis NB, particularly NB with a high ratio of NT-3:TrkC expression.

These results also demonstrate that compound capable of antagonizing TrkC-NT3 bound can be used as potential drug for the treatment and/or the prevention of cancer which results from an alteration of the TrkC/NT-3 receptor/ligand pair apoptotic activity in the cells, particularly neuroblastoma. These compounds antagonizing TrkC-NT3 bound induce the apoptotic death of these cancers cells. Together, these data all support the view that a fraction of NB shows an autocrine production of NT-3 associated with an increased NT-3/TrkC ratio. This elevated NT-3/TrkC ratio likely confers a selective advantage acquired by the cancer cells in settings of limited/no NT-3.

Here, we show that autocrine NT-3 expression is a mechanism developed by a large fraction of tumor cells to bypass TrkC-induced cell death that would occur in regions of limited NT-3 concentrations. Interestingly, this dependence on NT-3 presence appears specific for TrkC and is not involving other Trk receptors—i.e., TrkA or TrkB—as a dominant negative of TrkC is sufficient to turn down this dependence (FIG. 20A). As a consequence, NT-3 high expression constitutes a new marker for NB patients that could putatively respond to a treatment based on cell death induction via disruption of the NT-3/TrkC interaction.

The in vitro cell death effect and the in vivo anti-tumor effect of a blocking antibody on NT-3 expressing tumor cells call for a larger screen of cancers that could be responsive to such a therapeutic approach. Thus, it is clear from these results that a treatment based on inhibition of the interaction between NT-3 and its dependence receptor TrkC, by blocking either NT-3 or TrkC, can, as a first line treatment or in combination with standard chemotherapy, benefit to the large fraction of the patients suffering from aggressive NB with high NT-3 levels.

### REFERENCES

1. Levi-Montalcini, R & Angeletti, P U. (1963) Dev Biol 7, 653-659.
2. Huang, E J & Reichardt, L F. (2001) Annu Rev Neurosci 24, 677-736.
3. Kaplan, D R & Miller, F D. (2000) Curr Opin Neurobiol 10, 381-391.
4. Thibert, C, Teillet, M A, Lapointe, F, Mazelin, L, Le Douarin, N M & Mehlen, P. (2003) Science 301, 843-846.
5. Mazelin, L, Bernet, A, Bonod-Bidaud, C, Pays, L, Arnaud, S, Gespach, C, Bredesen, D E, Scoazec, J Y & Mehlen, P. (2004) Nature 431, 80-84.
6. Mehlen, P, Rabizadeh, S, Snipas, S J, Assa-Munt, N, Salvesen, G S & Bredesen, D E. (1998) Nature 395, 801-804.
7. Bordeaux, M C, Forcet, C, Granger, L, Corset, V, Bidaud, C, Billaud, M, Bredesen, D E, Edery, P & Mehlen, P. (2000) EMBO J 19, 4056-4063.
8. Llambi, F, Causeret, F, Bloch-Gallego, E & Mehlen, P. (2001) EMBO J 20, 2715-2722.
9. Thornberry, N A, Rano, T A, Peterson, E P, Rasper, D M, Timkey, T, Garcia-Calvo, M, Houtzager, V M, Nordstrom, P A, Roy, S & Vaillancourt, J P, et al. (1997) J Biol Chem 272, 17907-17911.
10. Patel, T D, Kramer, I, Kucera, J, Niederkofler, V, Jessell, T M, Arber, S & Snider, WD. (2003) Neuron 38, 403-416.
11. Forcet, C, Ye, X, Granger, L, Corset, V, Shin, H, Bredesen, D E & Mehlen, P. (2001) Proc Natl Acad Sci USA 98, 3416-3421.
12. Ockel, M, Lewin, G R & Barde, Y A. (1996) Development (Cambridge, UK) 122, 301-307.
13. Ringstedt, T, Kucera, J, Lendahl, U, Ernfors, P & Ibanez, C F. (1997) Development (Cambridge, UK) 124, 2603-2613.
14. Wright, D E, Zhou, L, Kucera, J & Snider, W D. (1997) Neuron 19, 503-517.
15. Crowley, C, Spencer, S D, Nishimura, M C, Chen, K S, Pitts-Meek, S, Armanini, M P, Ling, L H, McMahon, S B, Shelton, D L & Levinson, A D, et al. (1994) Cell 76, 1001-1011.
16. Minichiello, L, Piehl, F, Vazquez, E, Schimmang, T, Hokfelt, T, Represa, J & Klein, R. (1995) Development (Cambridge, UK) 121, 4067-4075.
17. Ernfors, P, Lee, K F & Jaenisch, R. (1994) Nature 368, 147-150.
18. Tessarollo, L, Tsoulfas, P, Donovan, M J, Palko, M E, Blair-Flynn, J, Hempstead, B L & Parada, L F. (1997) Proc Natl Acad Sci USA 94, 14776-14781.
19. Tessarollo, L, Vogel, K S, Palko, M E, Reid, S W & Parada, L F. (1994) Proc Natl Acad Sci USA 91, 11844-11848.
20. Farinas, I, Wilkinson, G A, Backus, C, Reichardt, L F & Patapoutian, A. (1998) Neuron 21, 325-334.
21. Farinas, I, Yoshida, C K, Backus, C & Reichardt, L F. (1996) Neuron 17, 1065-1078.
22. Davies, A M, Minichiello, L & Klein, R. (1995) EMBO J 14, 4482-4489.
23. ElShamy, W M & Ernfors, P. (1996) Development (Cambridge, UK) 122, 2405-2414.
24. White, F A, Silos-Santiago, I, Molliver, D C, Nishimura, M, Phillips, H, Barbacid, M & Snider, W D. (1996) J Neurosci 16, 4662-4672.
25. Matsunaga, E, Tauszig-Delamasure, S, Monnier, P P, Mueller, BK, Strittmatter, S M, Mehlen, P & Chedotal, A. (2004) Nat Cell Biol 6, 749-755.
26. Lee, R, Kermani, P, Teng, K K & Hempstead, B L. (2001) Science 294, 1945-1948.
27. Nykjaer, A, Lee, R, Teng, K K, Jansen, P, Madsen, P, Nielsen, M S, Jacobsen, C, Kliemannel, M, Schwarz, E & Willnow, T E, et al. (2004) Nature 427, 843-848.
28. Raoul, C, Estevez, A G, Nishimune, H, Cleveland, D W, de Lapeyriere, O, Henderson, C E, Haase, G & Pettmann, B. (2002) Neuron 35, 1067-1083. [CrossRef][ISI][Medline].
29. Salvesen, G S & Duckett, C S. (2002) Nat Rev Mol Cell Biol 3, 401-410.
30. Fernando, P, Kelly, J F, Balazsi, K, Slack, R S & Megeney, L A. (2002) Proc Natl Acad Sci USA 99, 11025-11030.
31. Campbell, D S & Holt, C E. (2003) Neuron 37, 939-952.
32. Grotzer, M A, Janss, A J, Phillips, P C & Trojanowski, J Q. (2000) Klin Padiatr 212, 196-199.
33. Kim, J Y, Sutton, M E, Lu, D J, Cho, T A, Goumnerova, L C, Goritchenko, L, Kaufman, J R, Lam, K K, Billet, A L & Tarbell, N J, et al. (1999) Cancer Res 59, 711-719.
34. Yu, L Y, Jokitalo, E, Sun, Y F, Mehlen, P, Lindholm, D, Saarma, M & Arumae, U. (2003) J Cell Biol 163, 987-997.
35. Moshnyakov, M, Arumae, U & Saarma, M. (1996) Brain Res Mol Brain Res 43, 141-148.
36. Tauszig-Delamasure, S., Yu, L. Y., Cabrera, J. R., Bouzas-Rodriguez, J., Mermet-Bouvier, C., Guix, C., Bordeaux, M. C., Arumae, U., and Mehlen, P. (2007). Proc Natl Acad Sci USA 104, 13361-13366.
37. Mehlen, P., and Thibert, C. (2004). Cell Mol Life Sci 61, 1854-1866.
38. Stupack, D. G., Teitz, T., Potter, M. D., Mikolon, D., Houghton, P. J., Kidd, V. J., Lahti, J. M., and Cheresh, D. A. (2006). Nature 439, 95-99.
39. Ghoumari, A. M., Wehrle, R., Bernard, O., Sotelo, C., and Dusart, I. (2000). Eur J Neurosci 12, 2935-2949.

1. A method for selecting a compound for the prevention or the treatment of cancer, wherein said method comprises the following steps of:
a) having a medium containing neurotrophin 3, or a fragment thereof, and a TrkC receptor, or a fragment thereof, wherein:
said neurotrophin 3, or a fragment thereof, and said TrkC receptor, or a fragment thereof, is able to specifically interact together to form a binding pair, and/or
said neurotrophin 3, or a fragment thereof, is able to induce the dimerization or multimerization of said TrkC receptor, or a fragment thereof, particularly the intracellular domain of said TrkC receptor;
b) contacting said medium with the compound to be tested;
c) measuring the inhibition of the interaction between neurotrophin 3, or a fragment thereof, and said TrkC receptor, or a fragment thereof, and/or
determine whether said compound inhibit the dimerization or multimerization of said TrkC receptor, or a fragment thereof, particularly the dimerization of the intracellular domain of said TrkC receptor; and
d) selecting said compound if:
the measuring in step c) demonstrates a significantly inhibition of the interaction between neurotrophin 3, or a fragment thereof, and TrkC receptor, or a fragment thereof, in presence of said compound, and/or
the determination in step c) demonstrates a significantly inhibition of the dimerization or multimerization of said TrkC receptor, or a fragment thereof, in presence of said compound, particularly the dimerization of the intracellular domain of said TrkC receptor.

2. The method according to claim 1, wherein said cancer to be prevented or treated is a cancer wherein tumoral cells express or overexpress neurotrophin 3 or express a high ratio neurotrophin 3/TrkC receptor.

3. The method according to claim 1, wherein said cancer to be prevented or treated is neuroblastoma.

4. The method according to claim 1, wherein said cancer to be prevent or treated is a metastatic cancer or an aggressive cancer, particularly a cancer having a poor prognosis.

5. The method according to claim 1, wherein at step a) said TrkC receptor is the human TrkC receptor or a fragment thereof, particularly a fragment containing at least the extracellular domain of the TrkC receptor.

6. The method according to claim 5, wherein said extracellular fragment comprises at least the N-terminal fragment containing the first 429 amino acid residues of the humant TrkC or of a natural variant thereof having at least 95% identity with the amino acid sequence depicted in Genbank A. N. AAB33111 dated Jul. 27, 1995.

7. The method according to claim 1, wherein at step a):
said TrkC receptor fragment comprises or is the extracellular domain of the TrkC receptor, or part thereof able to interact with neurotrophin 3; and/or
said TrkC receptor fragment comprises or is the intracellular domain of the TrkC receptor, or part thereof able to dimerize or multimerize in presence of neurotrophin 3.

8. The method according to claim 1, wherein at step a) said neurotrophin 3 or/and said TrkC receptor are from mammal, particularly from mouse, rat or human.

9. The method according to claim 1, wherein said neurotrophin 3 or/and said TrkC receptor and/or the compound to be tested is labelled by a marker able to be directly or indirectly measured.

10. The method according to claim 1, wherein at step c):
the measure of the inhibition of the interaction between neurotrophin 3, or a fragment thereof, and said TrkC receptor, or a fragment thereof, is carried out by immunoassay (particularly by ELISA or by Immunoradiometric Assay (IRMA)), by Scintillation Proximity Assay (SPA) or by Fluorescence Resonance Energy Transfer (FRET); and/or
the dimerization or multimerization, or its inhibition, of said TrkC receptor, or fragment thereof, particularly the intracellular domain, is carried out by immunoprecipitation or FRET.

11. The method according to claim 1, wherein at step a) said medium contains cells which express at their surface membrane an endogenous TrkC receptor or a recombinant TrkC receptor, particularly at least the extracellular domain of a recombinant TrkC receptor.

12. The method according to claim 11, wherein at step a) said medium contains cells which express recombinant TrkC receptor.

13. The method according to claim 11, wherein at step a) said medium contains tumoral cells which express endogenously said TrkC receptor at their membrane surface and which express or overexpress neurotrophin 3, and wherein at step c) the inhibition of the interaction between neurotrophin 3 and its TrkC receptor in presence of the compound to be tested, is measured by the apoptosis or cells death induced by the presence of the compound to be tested.

14. The method according to claim 13, wherein at step a) said medium contains metastatic tumoral cells, particularly cells selected from the group consisting of IMR32 cells, CM-Ge1 cells and CLb-Vol cells.

15. A method according to claim 1, for selecting a compound for the prevention or the treatment of cancer, wherein said method comprises the following steps of:
a) having a medium containing a mammal cell expressing an endogenous or a recombinant TrkC receptor, or a fragment thereof comprising at least its intracellular domain, preferably a tumor cell, more preferably a cell presenting dimerization or multimerization of its TrkC receptor intracellular domain or a cell wherein its TrkC receptor intracellular domain is able to dimerize or multimerize in presence of neurotrophin 3;
b) contacting said medium with the compound to be tested, optionally the medium further containing neurotrophin 3, or a fragment thereof able to interact with the extracellular domain of the TrkC receptor;
c) determine whether the dimerization or multimerization of said TrkC receptor intracellular domain is inhibited in presence of said compound to be tested;
d) optionally, determine whether the presence of the compound to be tested induces the cell death of said mammal cell; and
e) selecting said compound if the determination in step c) demonstrates a significant inhibition of the dimerization or multimerization of the intracellular domain of said TrkC receptor and/or if the determination in step d) demonstrates the cell death of said mammal cell.

16. An in vitro method for predicting the presence of a metastatic, an aggressive or a poor prognosis cancer in a patient having a primary tumor from a biopsy of said patient containing primary tumors cells, said method comprising the following step of:
(a) measuring of the neurotrophin 3 expression level in said biopsy or the ratio between the neurotrophin 3 expression level and the TrkC receptor expression level in said biopsy.

17. The method according to claim 16, wherein an increase of the neurotrophin 3 expression level in said biopsy, compared with expression of neurotrophin 3 in a non-metastatic primary tumor biopsies or in a non-aggressive cancer biopsies is significant of the presence of a metastatic cancer or an aggressive cancer.

18. The method according to claim 16, wherein a ratio superior to 2 between neurotrophin 3 expression in the biopsy to be tested and in the non-metastatic reference biopsy is significant of the presence of a metastatic cancer.

19. A method for determining in vitro the efficiency of an anti-cancer treatment for a patient or for in vitro selecting patients who are susceptible to respond to a specific anti-cancer treatment based on the inhibition of the NT-3:TrkC bound, said method comprising the following step of:
(a) obtaining a primary tumor biopsy of said treated patient; and
(b) measuring of the neurotrophin 3 expression level in said biopsy,
wherein the efficiency of said anti-cancer treatment is correlated with the decrease of the amount of the neurotrophin 3 expression level measured in said biopsy, or
wherein the selected patients who are susceptible to respond to said specific anti-cancer treatment are patients wherein the amount of the neurotrophin 3 expression level measured in their biopsy before the treatment is significantly superior to the amount of the neurotrophin 3 expression level of a control patient, and, optionally, wherein the neurotrophin 3 expression level has been decreased after said specific treatment.

20. A method according to claim 19, wherein said cancer induced an overexpression of neurotrophin 3 and/or is a metastatic or an aggressive cancer.

21. The method according to claim 16, wherein the measured neurotrophin 3 expression product is the RNA encoding neurotrophin 3, particularly measured by a quantitative real time reverse PCR method.

22. The method according to claim 16, wherein the expression level of neurotrophin 3 which is measured is the measuring of the neurotrophin 3 protein level, particularly by a method using specific antibodies able to specifically recognize said neurotrophin 3 protein.

23. The method according to claim 16, wherein the primary tumor is a primary tumor of neuroblastoma.

24. A kit for the selection of a compound for the prevention or the treatment of cancer, wherein said kit comprises:
a TrkC receptor protein, or a fragment thereof able to specifically interact with the neurotrophin 3 protein to form a binding pair, preferably recombinant protein; and
neurotrophin 3 protein, or a fragment thereof able to specifically interact with said TrkC receptor protein to form a binding pair, preferably recombinant protein.

25. A kit for the selection of a compound for the prevention or the treatment of cancer, wherein said kit comprises:
tumoral cells which express TrkC receptor and which express or overexpress neurotrophin 3, particularly cells from tumoral cell line, preferably selected from the group consisting of established neuroblastoma cell, preferred are selected from CLB-Ge1 and IMR-32 cell; and, optionally,
neurotrophin 3 protein, or a fragment thereof able to specifically interact with said TrkC receptor protein to form a binding pair, preferably a recombinant neurotrophin 3 protein.

26. A compound selected from the group consisting of:
a compound selected by the method of claim 1;
a compound comprising an extracellular domain of TrkC receptor or fragment thereof able to specifically inhibit the interaction between the neurotrophin 3 and said TrkC receptor, and/or able to inhibit the dimerization or multimerization of said TrkC receptor, or a fragment thereof, particularly the intracellular domain of said TrkC receptor, or a soluble; TrkC receptor comprising at least the extracellular domain of the TrkC capable of binding the neurotrophin 3;
a monoclonal or polyclonal antibody directed specifically against neurotrophin 3 or TrkC receptor, particularly directed to the extracellular domain of said TrkC receptor or to the neurotrophin 3 fragment able to interact with the extracellular domain of said TrkC receptor; and
a siRNA nucleic acid (small interfering RNA) capable of inhibiting the expression of NT3 in cells, preferably in vivo,
as a medicament.

27. A method of treatment for inducing the apoptosis or the cell death of tumor cells which have acquired the selective advantage to escape neurotrophin 3 dependence receptors induced apoptosis, preferably by elevated neurotrophin 3 level, in a patient comprising administering a compound able to inhibit this selective advantage in a tumor cell in said patient in need thereof.

28. The method for the prevention or for the treatment of cancer in a patient comprising administering a compound according to claim 26 in said patient in need thereof.

29. A use of a compound according to claim 26 for the manufacture of a medicament for the prevention or the treatment of cancer in mammals, including man.

30. The method or the use according to claim 28, wherein said cancer is a metastatic, an aggressive cancer or a poor prognosis cancer.

31. The method according to claim 27, wherein said cancer is neuroblastoma.

32. The method according to claim 27, wherein the primary tumor cells of said cancer express or overexpress neurotrophin 3.

33. A use of the level of neurotrophin 3 expression as a marker for the identification of a metastatic, an aggressive cancer or a bad prognosis cancer in a patient.

34. The use according to claim 33, wherein said cancer is neuroblastoma.


SEQUENCE LISTING (32 sequences)

SEQ ID NO: 1 - DNA, 27 bp, artificial sequence
  Forward primer for subcloning Trk IC
caccatgaacaagtacggtcgacggtc

SEQ ID NO: 2 - DNA, 21 bp, artificial sequence
  Reverse primer for subcloning Trk IC
ctggacattcttggctagtgg

SEQ ID NO: 3 - DNA, 34 bp, artificial sequence
  D641N primer for obtaining TrkC mutants
gcgatgatccttgtgaatggacagccacgccagg

SEQ ID NO: 4 - DNA, 34 bp, artificial sequence
  D641N primer for obtaining TrkC mutants
cctggcgtggctgtccattcacaaggatcatcgc

SEQ ID NO: 5 - DNA, 32 bp, artificial sequence
  D495N primer for obtaining TrkC mutants
acaccttcatcgctgaatgctgggccggatac

SEQ ID NO: 6 - DNA, 32 bp, artificial sequence
  D495N primer for obtaining TrkC mutants
gtatccggcccagcattcagcgatgaaggtgt

SEQ ID NO: 7 - DNA, 28 bp, artificial sequence
  D679N primer for obtaining TrkC mutants
ctttgtgcaccgaaacctggccaccagg

SEQ ID NO: 8 - DNA, 28 bp, artificial sequence
  D679N primer for obtaining TrkC mutants
cctggtggccaggtttcggtgcacaaag

SEQ ID NO: 9 - DNA, 26 bp, artificial sequence
  496-642 fragment forward primer for cloning constructs expressing the different TrkC domains
caccatggctgggccggatacagtgg

SEQ ID NO: 10 - DNA, 24 bp, artificial sequence
  496-642 fragment reverse primer for cloning constructs expressing the different TrkC domains
tcaatccacaaggatcatcgcatc

SEQ ID NO: 11 - DNA, 26 bp, artificial sequence
  496-825 fragment forward primer for cloning constructs expressing the different TrkC domains
caccatggctgggccggatacagtgg

SEQ ID NO: 12 - DNA, 22 bp, artificial sequence
  496-825 fragment reverse primer for cloning constructs expressing the different TrkC domains
ctagccaagaatgtccaggtag

SEQ ID NO: 13 - DNA, 34 bp, artificial sequence
  642-825 fragment forward primer for cloning constructs expressing the different TrkC domains
ggcctggcgtggctgtcaatccacaaggatcatc

SEQ ID NO: 14 - DNA, 22 bp, artificial sequence
  642-825 fragment reverse primer for cloning constructs expressing the different TrkC domains
ctagccaagaatgtccaggtag

SEQ ID NO: 15 - DNA, 28 bp, artificial sequence
  Forward primer for subcloning human TrkC
cgcaccatggatgtctctctttgcccag

SEQ ID NO: 16 - DNA, 31 bp, artificial sequence
  Reverse primer for subcloning human TrkC
gcgtctagactagccaagaatgtccaggtag

SEQ ID NO: 17 - RNA, 21 nt, artificial sequence
  1S primer for targetting the 3′UTR region of TrkC to invalidate endogenous TrkC
ucucaacuccuuucuuccauu

SEQ ID NO: 18 - RNA, 21 nt, artificial sequence
  1AS primer for targetting the 3′UTR region of TrkC to invalidate endogenous TrkC
uggaagaaaggaguugagauu

SEQ ID NO: 19 - RNA, 21 nt, artificial sequence
  2S primer for targetting the 3′UTR region of TrkC to invalidate endogenous TrkC
cucaagugccugcuacacaua

SEQ ID NO: 20 - RNA, 21 nt, artificial sequence
  2AS primer for targetting the 3′UTR region of TrkC to invalidate endogenous TrkC
uguguagcaggcacuugagua

SEQ ID NO: 21 - RNA, 21 nt, artificial sequence
  3S primer for targetting the 3′UTR region of TrkC to invalidate endogenous TrkC
gcauuuauacucuguugccuc

SEQ ID NO: 22 - RNA, 21 nt, artificial sequence
  3AS primer for targetting the 3′UTR region of TrkC to invalidate endogenous TrkC
ggcaacagaguauaaaugcuc

SEQ ID NO: 23 - DNA, 20 bp, artificial sequence
  TrkC forward primer
agctcaacagccagaacctc

SEQ ID NO: 24 - DNA, 20 bp, artificial sequence
  TrkC reverse primer
aacagcgttgtcaccctctc

SEQ ID NO: 25 - DNA, 20 bp, artificial sequence
  NT-3 forward primer
gaaacgcgatgtaaggaagc

SEQ ID NO: 26 - DNA, 20 bp, artificial sequence
  NT-3 reverse primer
ccagcccacgagtttattgt

SEQ ID NO: 27 - DNA, 21 bp, artificial sequence
  HPRT forward primer
tgacactggcaaaacaatgca

SEQ ID NO: 28 - DNA, 21 bp, artificial sequence
  HPRT reverse primer
ggtccttttcaccagcaagct

SEQ ID NO: 29 - DNA, 21 bp, artificial sequence
  Alu sequence forward primer
acgcctgtaatcccagcactt

SEQ ID NO: 30 - DNA, 19 bp, artificial sequence
  Alu sequence reverse primer
tcgcccaggctggagtgca

SEQ ID NO: 31 - DNA, 24 bp, artificial sequence
  GAPDH forward primer
gaggaaaggtcgcctggtggatcg

SEQ ID NO: 32 - DNA, 24 bp, artificial sequence
  GAPDH reverse primer
ggtgaggacaagcagtgaggaacg